(19) United States
(12) Patent Application Publication
(10) Pub. No.: US 2023/0256236 A1
(43) Pub. Date: Aug. 17, 2023

(54) TRANSFORMABLE NEEDLE FOR ELECTROPORATION

(71) Applicant: OncoSec Medical Incorporated, Pennington, NJ (US)

(72) Inventors: John F. RODRIGUEZ, Vista, CA (US); Jose GARCIA, Pennington, NJ (US); Brandon Dang PHUNG, San Diego, CA (US)

(21) Appl. No.: 18/011,115

(22) PCT Filed: Jun. 24, 2021
(86) PCT No.: PCT/US21/38914

§ 371 Date: Dec. 16, 2022

Related U.S. Application Data
(60) Provisional application No. 62/705,373, filed on Jun. 24, 2020.

(51) Int. Cl. A61N 1/05; A61N 1/32

(52) U.S. Cl. CPC A61N 1/0502; A61N 1/327

(57) ABSTRACT

Provided herein are systems, methods, and apparatus for electroporation. An example transformable needle includes a first electroporation member defining a first expandable portion, a distal end, and a proximal end, with the first expandable portion defining a first conductive portion. The transformable needle also includes a second electroporation member defining a second expandable portion, a distal end, and a proximal end, with the second expandable portion defining a second conductive portion. The transformable needle further includes a needle casing defining a distal end. The first expandable portion and the second expandable portion are each configured to move between a retracted position and a deployed position. A distance between the first conductive portion of the first expandable portion and the second conductive portion of the second expandable portion is greater in the deployed position than in the retracted position.

### CROSS-REFERENCE TO RELATED APPLICATION

This application claims priority to and the benefit of U.S. Provisional Application No. 62/705,373, filed Jun. 24, 2020, which is incorporated herein by reference in its entirety.

### BACKGROUND

Electrical fields may be used to create pores in cells through a process known as electroporation to increase the permeability of target cells and administer various localized treatments to a patient. There is a need for electroporation therapy in difficult to reach areas of the body, such as to treat tumors within the lungs, and there is a need to provide a large treatment area while still being able to fit the electroporation devices into these difficult to reach areas. There is also a need to administer a variety of treatment agents and therapies with a high degree of precision and minimal invasiveness.

Through applied effort, ingenuity, and innovation, many of these identified problems have been solved by developing solutions that are included in embodiments of the present invention, many examples of which are described in detail herein.

### BRIEF SUMMARY

Disclosed herein are electroporation systems, applicators, associated methods of treatment and use, and associated apparatus. The following presents a simplified summary in order to provide a basic understanding of some aspects of the present disclosure. This summary is not an extensive overview and is intended to neither identify key or critical elements nor delineate the scope of such elements. Its purpose is to present some concepts of the described features in a simplified form as a prelude to the more detailed description that is presented later.

In an example embodiment, a transformable needle for an electroporation applicator is provided. The transformable needle includes a first electroporation member defining a first expandable portion, a distal end, and a proximal end. The first expandable portion is defined between the distal end and the proximal end and the first expandable portion defines a first conductive portion. The transformable needle also includes a second electroporation member defining a second expandable portion, a distal end, and a proximal end. The second expandable portion is defined between the distal end and the proximal end and the second expandable portion defines a second conductive portion. The transformable needle further includes a needle casing defining a distal end. The first electroporation member and the second electroporation member are attached to the needle casing. The distal end of the first electroporation member and the distal end of the second electroporation member each are configured to be stationary relative to the needle casing. The first expandable portion and the second expandable portion are each configured to move between a retracted position and a deployed position. A distance between the first conductive portion of the first expandable portion and the second conductive portion of the second expandable portion is greater in the deployed position than in the retracted position. In at least the deployed position, the first conductive portion of the first expandable portion and the second conductive portion the second expandable portion are electrically isolated from each other in the transformable needle, such that application of a voltage between the first conductive portion and the second conductive portion is configured to create an electric field in at least a portion of a target tissue.

In some embodiments, the needle casing defines a first electroporation member channel and a second electroporation member channel. In such an embodiment, at least a portion of the first electroporation member is received by the first electroporation member channel and at least a portion of the second electroporation member is received by the second electroporation member channel. In some embodiments, the first electroporation member and the second electroporation member each defines an attachment mechanism at the distal end of the given electroporation member. In such an embodiment, the attachment mechanism is configured to removably attach the given electroporation member to a distal end of the needle casing.

In some embodiments, the transformable needle also includes a needle tip defined at the distal end of the needle casing. In some embodiments, in an instance the first expandable portion and the second expandable portion are in the deployed position, the first expandable portion and the second expandable portion define a diamond shape. In such an embodiment, the first conductive portion of the first expandable portion and the second conductive portion of the second expandable portion are parallel. In some embodiments, the first expandable portion and the second expandable portion are each configured to have a transition point. In such an embodiment, in an instance the first expandable portion and the second expandable portion are in the deployed position the transition point expands and at least one of the distal end or proximal end of the respective expandable portion remains fixed.

In some embodiments, the first expandable portion and the second expandable portion are each configured to have a hinge point. In such an embodiment, in an instance the first expandable portion and the second expandable portion are in the deployed position the hinge point expands and at least one of the distal end or proximal end of the respective expandable portion remains fixed. In some embodiments, the first expandable portion also includes a first non-conductive portion and the second expandable portion also includes a second non-conductive portion. In some embodiments, the first non-conductive portion and the second non-conductive portion are portions of the respective expandable portion coated with a non-conductive material.

In some embodiments, the transformable needle also includes a deployment mechanism in operably communication with the proximal end of the first expandable portion and the second expandable portion. In such an embodiment, the deployment mechanism being configured to allow the first expandable portion and the second expandable portion to move between the retracted position and the deployed position. In some embodiments, the deployment mechanism is configured to move the first electroporation member and the second electroporation member to a position in which the given expandable portion aligns with a given electroporation aperture. In some embodiments, the transformable needle also includes a deployment mechanism switch configured to move the deployment mechanism between the retracted position and the deployed positioned.

In some embodiments, at least one of the first expandable portion or the second expandable portion comprises nitinol. In some embodiments, the needle casing is an exterior needle casing. In such an embodiment, the first electroporation member and the second electroporation member are disposed within the exterior needle casing. In some embodiments, the transformable needle also includes a drug delivery channel disposed within the exterior needle casing between the two electroporation members. In some embodiments, the drug delivery channel, the needle casing, and the distal ends of each of the electroporation members are all stationary relative to each other during operation.

In some embodiments, the exterior needle defines a first electroporation aperture and a second electroporation aperture. In such an embodiment, the first expandable portion and the second expandable portion are configured to align with the first electroporation aperture and the second electroporation aperture respectively at least in an instance in which the first electroporation member and the second electroporation member are in the deployed position. In some embodiments, the drug delivery channel includes one or more delivery side ports configured to be aligned with at least one of the first expandable portion or the second expandable portion. In such an embodiment, the delivery side ports are configured to be exposed in an instance the first electroporation member and the second electroporation member are in the deployed position.

In some embodiments, the drug delivery channel includes one or more delivery side ports configured to be aligned with the first electroporation aperture or the second electroporation aperture. In some embodiments, the one or more delivery side ports are configured to be fixably aligned with the first electroporation aperture or the second electroporation aperture. In some embodiments, one or more delivery side ports are configured to be aligned with at least one of the first expandable portion or the second expandable portion. In such an embodiment, the delivery side ports are configured to be exposed in an instance the first electroporation member and the second electroporation member are in the deployed position.

In another example embodiment, a method of using a transformable needle discussed herein is provided. The method includes moving the first expandable portion and the second expandable portion between a retracted position and a deployed position. A distance between the first conductive portion of the first expandable portion and the second conductive portion of the second expandable portion is greater in the deployed position than in the retracted position. The method also includes applying voltage between the first conductive portion and the second conductive portion. The applying voltage between the first conductive portion and the second conductive portion creates an electric field in at least a portion of the target tissue.

In some embodiments, the needle casing defines a first electroporation member channel and a second electroporation member channel. In such an embodiment, at least a portion of the first electroporation member is received by the first electroporation member channel and at least a portion of the second electroporation member is received by the second electroporation member channel. In some embodiments, the first electroporation member and the second electroporation member each defines an attachment mechanism at the distal end of the given electroporation member. In such an embodiment, the attachment mechanism is configured to removably attach the given electroporation member to a distal end of the needle casing. In some embodiments, the transformable needle also includes a needle tip defined at the distal end of the needle casing. In some embodiments, in an instance the first expandable portion and the second expandable portion are in the deployed position, the first expandable portion and the second expandable portion define a diamond shape. In such an embodiment, the first conductive portion of the first expandable portion and the second conductive portion of the second expandable portion are parallel.

In some embodiments, the first expandable portion and the second expandable portion are each configured to have a transition point. In such an embodiment, in an instance the first expandable portion and the second expandable portion are in the deployed position the transition point expands and at least one of the distal end or proximal end of the respective expandable portion remains fixed. In some embodiments, the first expandable portion and the second expandable portion are each configured to have a hinge point. In such an embodiment, in an instance the first expandable portion and the second expandable portion are in the deployed position the hinge point expands and at least one of the distal end or proximal end of the respective expandable portion remains fixed. In some embodiments, the first expandable portion also includes a first non-conductive portion and the second expandable portion further includes a second non-conductive portion. In some embodiments, the first non-conductive portion and the second non-conductive portion are portions of the respective expandable portion coated with a non-conductive material.

In some embodiments, the method also includes restricting, via a deployment mechanism, the movement of the first expandable portion and the second expandable portion in an instance the first expandable portion and the second expandable portion are in the retracted position. In such an embodiment, the deployment mechanism in operably communication with the proximal end of the first expandable portion and the second expandable portion. In some embodiments, the deployment mechanism is configured to move the first electroporation member and the second electroporation member to a position in which the given expandable portion aligns with a given electroporation aperture. In some embodiments, the method also includes moving, via a deployment mechanism switch, the deployment mechanism between the retracted position and the deployed positioned. In such an embodiment, the deployment mechanism switch is configured to move the deployment mechanism between the retracted position and the deployed positioned. In some embodiments, at least one of the first expandable portion or the second expandable portion includes nitinol. In some embodiments, the needle casing is an exterior needle casing. In such an embodiment, the first electroporation member and the second electroporation member are disposed within the exterior needle casing.

In some embodiments, the transformable needle also includes a drug delivery channel disposed within the exterior needle casing between the two electroporation members. In some embodiments, the drug delivery channel, the needle casing, and the distal ends of each of the electroporation members are all stationary relative to each other during operation. In some embodiments, the exterior needle defines a first electroporation aperture and a second electroporation aperture. The first expandable portion and the second expandable portion are configured to align with the first electroporation aperture and the second electroporation aperture respectively at least in an instance in which the first electroporation member and the second electroporation member are in the deployed position.

In some embodiments, the drug delivery channel includes one or more delivery side ports configured to be aligned with at least one of the first expandable portion or the second expandable portion. In such an embodiment, the delivery side ports are configured to be exposed in an instance the first electroporation member and the second electroporation member are in the deployed position. In some embodiments, the drug delivery channel includes one or more delivery side ports configured to be aligned with the first electroporation aperture or the second electroporation aperture. In some embodiments, the one or more delivery side ports are configured to be fixably aligned with the first electroporation aperture or the second electroporation aperture. In some embodiments, one or more delivery side ports are configured to be aligned with at least one of the first expandable portion or the second expandable portion. In such an embodiment, the delivery side ports are configured to be exposed in an instance the first electroporation member and the second electroporation member are in the deployed position.

The above summary is provided merely for purposes of summarizing some example embodiments to provide a basic understanding of some aspects of the disclosure. Accordingly, it will be appreciated that the above-described embodiments are merely examples and should not be construed to narrow the scope or spirit of the disclosure in any way. It will be appreciated that the scope of the disclosure encompasses many potential embodiments in addition to those here summarized, some of which will be further described below.

### BRIEF DESCRIPTION OF THE SEVERAL VIEWS OF THE DRAWINGS

Having thus described embodiments of the invention in general terms, reference will now be made to the accompanying drawings, which are not necessarily drawn to scale, and wherein:

FIG. 1 shows a block diagram of an electroporation system in accordance with some embodiments;

FIG. 2 shows a generator and simplified applicator in accordance with some embodiments;

FIG. 3 shows an endoscope in accordance with some embodiments;

FIG. 4A shows a perspective view of an applicator having electrodes in a retracted position in accordance with some embodiments;

FIG. 4B shows a sectional view of the applicator having electrodes in a retracted position in accordance with some embodiments;

FIG. 5A illustrates an actuator/switch assembly to be inserted into the applicator, such as the one shown in FIGS. 4A and 4B, in accordance with an example embodiment of the present disclosure

FIG. 5B is a cutaway view of a Block 5B of the transformable needle shown in FIG. 5A in the deployed position in accordance with an example embodiment of the present disclosure;

FIG. 6A is an exterior view of a section of the transformable needle in the deployed position in accordance with an example embodiment of the present disclosure;

FIG. 6B is a cut-way view of a section of the transformable needle in the deployed position in accordance with an example embodiment of the present disclosure

FIG. 7A illustrates the distal end of the needle casing in accordance with an example embodiment of the present disclosure; and

FIG. 7B illustrates the distal end of the needle to be used in a transformable needle in an example embodiment of the present disclosure.

### DETAILED DESCRIPTION

Some embodiments of the present invention will now be described more fully hereinafter with reference to the accompanying drawings, in which some, but not all embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout.

Various embodiments discussed herein provide a transformable needle and method of using the same. The transformable needle of various embodiments, allows for a unitary needle that includes the electrodes to form a single common tip. In some embodiments, the transformable needle may allow for drug delivery and/or electroporation using only a single needle. Additionally, the transformable needle of various embodiments allows for the needle to quickly and easily move between a retracted position, such that the transformable needle can be inserted and/or removed from the body for use, and a deployed position, such that the intended target (e.g., a tumor) may be electroporated.

### System Overview

Disclosed herein are various electroporation systems, apparatus, and methods. In some embodiments, the electroporation systems, apparatus, and methods disclosed herein may be used in connection with minimally-invasive procedures involving inserting portions of an applicator into a patient via a narrow opening and, in some embodiments, administering various therapies and treatment agents therethrough. The systems, apparatus, and method used herein may be used to deliver any treatment agent (e.g., nucleic acid-based therapies) and apply any electroporation therapy viscerally. In some embodiments, the electroporation systems, apparatus, and methods disclosed herein may be used in connection with an insertion device.

As used herein, the term “insertion device” means any apparatus or structure capable of allowing a portion of an applicator to be inserted into a patient, for example via a cannula or other working channel. In some embodiments, the electroporation systems, apparatus, and methods disclosed herein may be used in connection with endoscopic devices and procedures to reach and treat remote tissues (e.g., visceral lesions, such as tumors) within a patient. In some embodiments, various types of endoscopic devices may be used along with the electroporation systems, apparatus, and methods disclosed herein depending on the particular location of the remote tissue, such as bronchoscopic devices, laparoscopic devices or other cannulated devices suitable for providing access to such remote tissues. Such endoscopic devices may be of any type, including for example either a flexible endoscopic instrument or a rigid endoscopic instrument (e.g., a trocar, such as for use in laparoscopic procedures), which may be selected based on the anticipated procedure and/or location of the remote tissue. In some embodiments, the electroporation systems, apparatus, and methods disclosed herein may be used to access lesions anywhere in or adjacent to the alimentary canal. In some embodiments, the electroporation systems, apparatus, and methods disclosed herein may be used to access lesions in the lungs. In some embodiments, the electroporation systems, apparatus, and methods disclosed herein may be used in connection with minimally invasive electroporation, one example being in connection with any such aforementioned endoscopic instrument.

In a variety of medical treatments, electroporation may be used to increase the permeability of cells by using electrical fields to create pores in biological cells without causing permanent damage (e.g., reversible electroporation). In some instances, the increased permeability of reversible electroporation may enable a contemporaneous treatment, such as drug administration or gene therapy, to be more effective because the treatment is better able to permeate the cells. During electroporation, a voltage may be applied across two or more electrodes to create an electric field therebetween. In some examples, the electrodes may be disposed on either side of, embedded within, or otherwise be positioned relative to, cell tissue that is then subjected to the electric field. The electric field creates the pores within the cell tissue which then allow the cell to be permeated by one or more treatment agents. Performance of electroporation with a low voltage generator as described herein is particularly advantageous in satisfying the conditions necessary to achieve reversible electroporation. Although tissue around the target site may have varying electric field thresholds, the application of low voltage is intended to, even among the extant range of threshold values, apply a voltage amount that is below such a threshold in order to minimize or avoid damage to the tissue during the electroporation procedure.

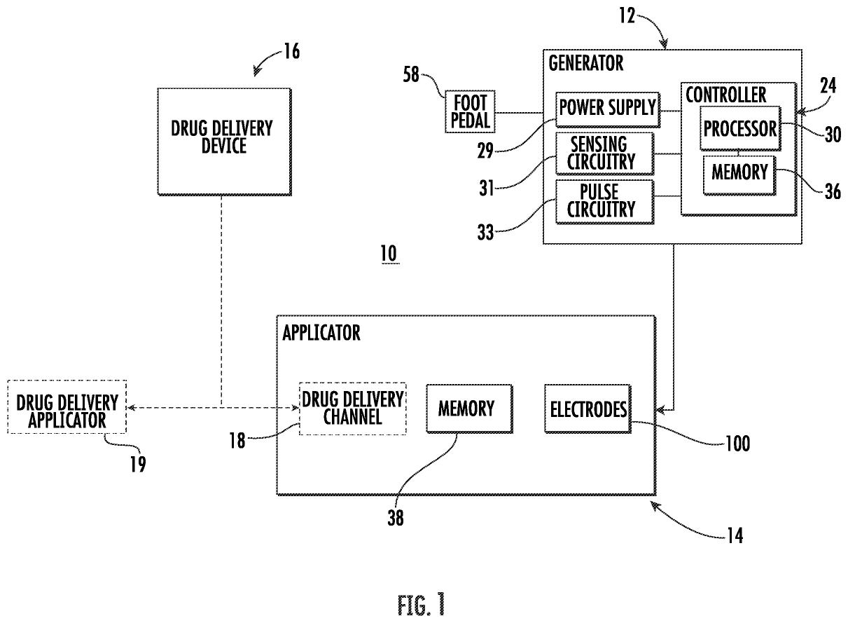

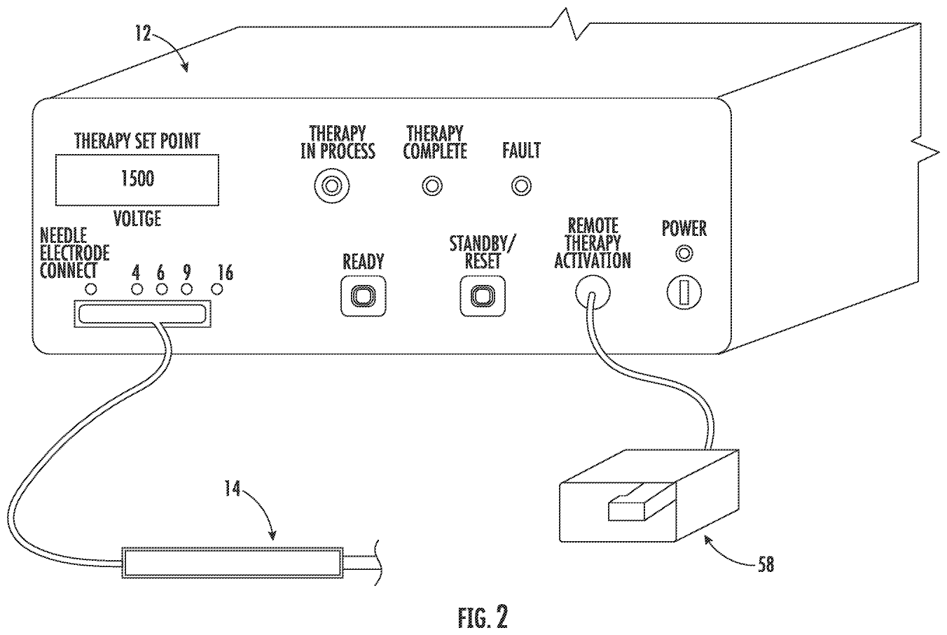

With reference to FIGS. 1-2, an example electroporation system 10 is shown. In the embodiment as illustrated, the system 10 includes a generator 12 for generating and delivering electrical signals to at least two electrodes 100 and an applicator 14 including the at least two electrodes.

### Example System Architecture

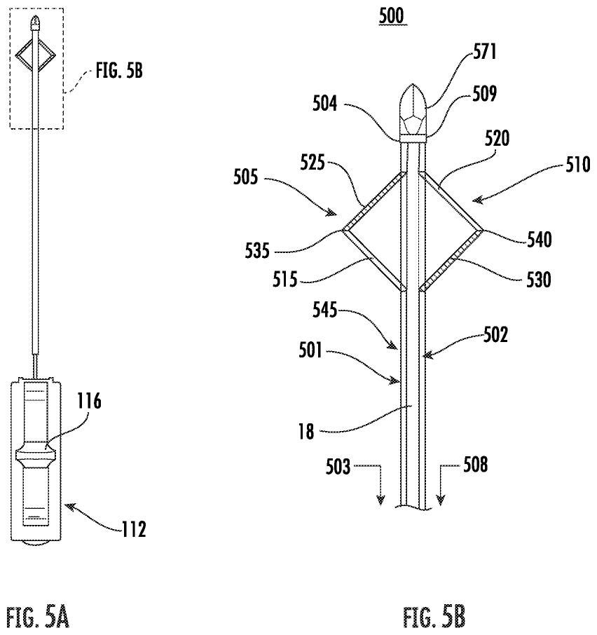

In some embodiments, the generator 12 and applicator 14 are controlled by one or more controllers 24, which includes at least a processor 30 and memory 36. In some embodiments, the controller 24 may be disposed in the generator 12 and may control the applicator 14 therewith. In various embodiments, the applicator 14 may be the transformable needle 500 shown in FIG. 5. In various embodiments, a drug delivery device 16 may be provided comprising one or more treatment agents. In various embodiments, the drug delivery device 16 may be configured to deliver the one or more treatment agents through a drug delivery channel 18 associated with the applicator, with at least a portion of the applicator being inserted into the working channel of an endoscope, (e.g., a flexible endoscope, a rigid endoscope, trocar, or the like). For example, the drug delivery device 16 may provide one or more treatment agents to the drug delivery channel 18, such that the one or more treatment agents may be provided through the injection ports in an instance in which the transformable needle 500 is in the deployed position. In embodiments in which the drug delivery device 16 requires electronic control, one or more controllers may operate the drug delivery device, and in embodiments in which the drug delivery device 16 has no electronic control, the drug delivery device may be manually operated (e.g., by depressing a syringe). In some embodiments, electronic control may be in the form of robotics, described elsewhere herein. In some embodiments, each of the generator 12, applicator 14, and drug delivery device 16 may have its own controller. In some embodiments, one or more of the controllers may be controlled by another controller (e.g., in a master-slave relationship). In some embodiments, each controller 24 may be embodied as a single device or as a distributed processing system, some or all of which may be remote from the respective device that it controls. Examples of an electroporation system and corresponding electronic control methods, signals, and apparatus; treatment agents; and therapies are described in U.S. Pat. Nos. 7,412,284 and 9,020,605, U.S. application Ser. No. 16/401,811, and International Application No. WO2016/161201, each of which is incorporated by reference herein in its entirety.

With continued reference to FIG. 1, in some embodiments, the generator 12 may be a low-voltage generator for administering the electroporation therapy and/or performing electrochemical impedance spectroscopy (EIS) as described herein. In some embodiments, the generator 12 may include pulse circuitry 33 configured to generate waveforms for excitation of the electrodes during electroporation. In some embodiments, the generator 12 is configured solely to perform electroporation therapy. In some embodiments, the generator 12 may include sensing circuitry 31 configured to receive signals from the electrodes 100 (e.g., EIS signals) and facilitate analysis of the properties of the target tissue. As described herein, in some embodiments, the generator 12 may control the pulses output from the pulse circuitry 33 in response to the sensed parameters of the target tissue and the treatment agent determined by the sensing circuitry 31. In embodiments of the system with sensing circuitry 31, the circuitry may be toggled to activate or deactivate control of the parameters of the electroporation therapy based on the analysis of the EIS signals received by the system. In this manner, if the circuitry is toggled off, the therapy will maintain a preset voltage and pulse duration (or a predetermined voltage and pulse duration pattern) irrespective of any variation in impedance reported to the system by the sensors.

Turning to the structure of the generator of the system, in some embodiments, the low voltage generator includes a digital board and a power generation board. The digital board may provide the central computing system by which signal processing, peripheral outputs, and safety features for the generator are implemented, while the power generation board contains all of the electrical components for pulse delivery during an electroporation treatment.

The digital board may include a microcontroller (MC), a digital-analog convertor (DAC), two analog-digital convertors (ADCs), resistor bank circuits, preamplifier circuits, and peripheral circuits. Each of these components contribute to the output of the device and signal processing for EIS. The MC also computes the software-based safety features to prevent delivery of unsafe therapy. Combined, the digital board may integrate both data acquisition components with the microcontroller unit to increase signal integrity by forgoing the cable assemblies between the two boards.

In some embodiments, the generator 12 may include a power supply 29 configured to receive power from the electrical mains and supply electrical energy to the system 10. In some embodiments, the generator 12 connects to the applicator via a wired connection. In some embodiments, at least one connection between the generator 12 and the applicator is a wireless connection. In some examples, the wireless connection may utilize low-energy communication with the respective elements being configured to send and receive signals. The low-energy communication technology may be Bluetooth®. In some embodiments, the generator may be a high voltage generator.

The processor 30 may be embodied in a number of different ways. For example, the processor 30 may be embodied as various processing means such as one or more of a microprocessor or other processing element, a coprocessor, a controller, or various other computing or processing devices including integrated circuits such as, for example, an ASIC (application specific integrated circuit), an FPGA (field programmable gate array), or the like. Although illustrated as a single processor, it will be appreciated that the processor 30 may comprise a plurality of processors in each device of the system or a single or plurality of centralized processors for multiple devices. The processor may be in operative communication with and may be configured to perform one or more functionalities for the devices of the electroporation system 10 as described herein. The processor may be embodied on a single computing device or distributed across a plurality of computing devices collectively configured to function as a controller 24. For example, a user device such as a smart phone, tablet, personal computer and/or the like may be configured to communicate with a detection device linked with the processor via means such as by Bluetooth™ communication or over a local area network. Additionally or alternatively, a remote server device may perform some of the operations described herein, such as processing data collected by any of the sensors, and providing or communicating resultant data to other devices accordingly.

In some example embodiments, the processor 30, may be configured to execute instructions stored in the memory 36 or otherwise accessible to the processor. As such, whether configured by hardware or by a combination of hardware and software, the processor 30 may represent entities (e.g., physically embodied in circuitry—in the form of processing circuitry) capable of performing operations according to embodiments of the present invention while configured accordingly. Thus, for example, when the processor 30 is embodied as an ASIC, FPGA, or the like, the processor 30 may be specifically configured hardware for conducting the operations described herein. Alternatively, as another example, when the processor 30 is embodied as an executor of software instructions, the instructions may specifically configure the processor 30 to perform one or more operations described herein.

In some embodiments, the applicator 14 may further include a memory 38 that stores information relating to the applicator. The controller 24 may interrogate the memory 38 of the applicator and identify the applicator and any necessary steps or instructions to execute electroporation based on the data stored in the memory 38. In this manner, the controller 24 may identify the applicator 14 before beginning electroporation. In some embodiments, the memory 38 may be disposed in the cable assembly.

In some example embodiments, the memory 36, 38 of the generator and applicator, respectively, may include one or more non-transitory memory devices such as, for example, volatile and/or non-volatile memory that may be either fixed or removable. In this regard, each memory 36, 38 may comprise non-transitory computer-readable storage media. It will be appreciated that while each memory 36, 38 is illustrated as a single memory in each device, each memory 36, 38 may comprise a plurality of memories in one or more devices or a single memory or centralized memory or plurality of memories for multiple devices. The centralized memory may be embodied on a single computing device or may be distributed across a plurality of computing devices. Each memory 36, 38 (or centralized memory(ies)) may be configured to store information, data, applications, computer program code, instructions and/or the like for enabling the electroporation system 10 to carry out various functions in accordance with one or more example embodiments.

Each memory 36, 38 (or any centralized memory or the like) may be configured to buffer input data for processing by the processor 30. Additionally or alternatively, such memory may be configured to store instructions for execution by the processor 30. In some embodiments, such memory may include one or more databases that may store a variety of files, contents, or data sets. For instance, among the contents of each memory 36, 38, applications may be stored for execution by the processor 30 to carry out the functionality associated with each respective application. As a further example, each memory 36, 38 may store data detected by a sensor(s) of the detection device, and/or application code for processing such data according to example embodiments. In some cases, each memory 36, 38 may be in communication with one or more of the processor 30, the electrodes 100, the generator 12, the drug delivery device 16, and/or other apparatus and sensors. In some embodiments, each memory 36, 38 may store step by step commands for specific surgical procedures that may be executed by the processor. For example, this may include details to navigate the applicator to a target site for a bronchoscopy. In a further example, such details may be used as commands for a robot to move the applicator to a target site and/or perform a procedure (in such an instance, a centralized memory or memories may be preferred, and such memory may even be included in the robot itself). This type of storage is also contemplated for other procedures as described elsewhere in the disclosure. In some embodiments, one or more of the memory 36, 38 may comprise an electrically erasable programmable read-only memory (EEPROM). In some embodiments, the applicator 14 memory 38 may include an EEPROM chip.

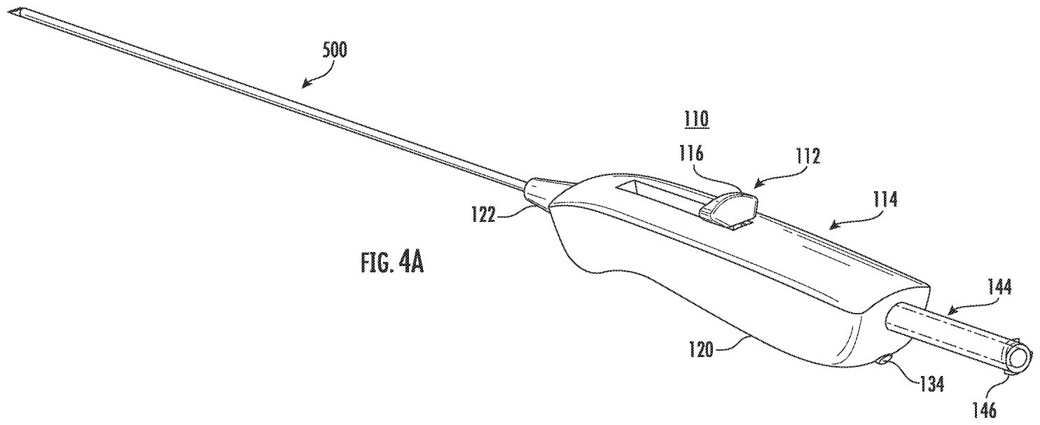

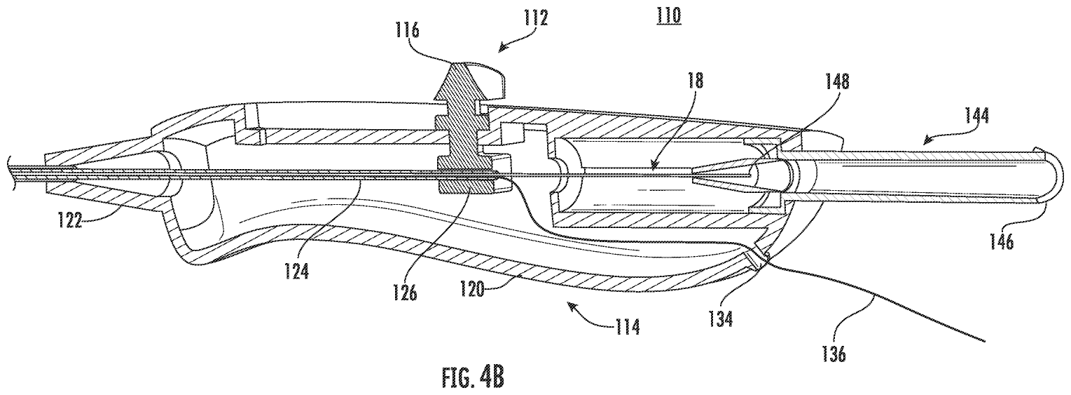

With reference to FIG. 2, an example generator 12 and simplified applicator 14 are shown for example purposes. The applicator 14 may include embodiments of the transformable needle described herein, such as the applicator 110 shown in FIG. 4A. The generator may generate electrical signals to electroporate the target tissues. The generator 12 may regulate the properties of the electrical signals (e.g., voltage, amplitude, frequency, duration, and the like) to cause reversible electroporation of the tissues without damaging the target tissues. In some embodiments, the generator 12 may include a foot pedal 58 for allowing a user to actuate and operate the generator and electroporation. The foot pedal 58 may be connected to the generator via a wired connection or via a low energy wireless connection, such as Bluetooth®. Where a wireless connection is used, each of the foot pedal 58 and the generator may include sensors to send and receive signals communicating changes in the status of the foot pedal 58. Operation of the generator may be aided or fully controlled by a robotic system. For example, a robotic arm may be configured to control the generator to achieve desired electrical parameters for electroporation. Examples of an electroporation system and corresponding electronic control methods, signals, and apparatus are described in U.S. Pat. Nos. 7,412,284 and 9,020,605; U.S. application Ser. No. 16/401,811; and International Application No. WO2016/161201, each of which is incorporated by reference herein in its entirety.

### Example Electroporation Applicator

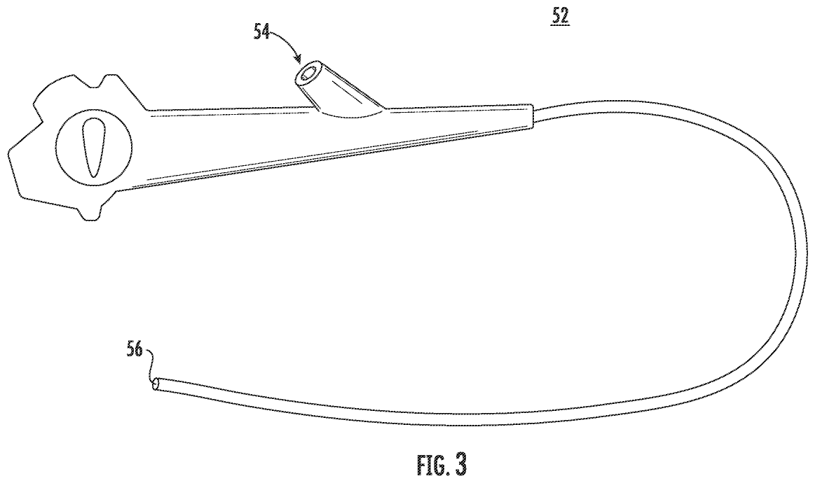

In some embodiments, the electroporation system 10 may be operable for use with access instrumentation, such as an endoscope or the like, while in some embodiments, the electroporation system may be insertable independently. Endoscopy involves inserting an endoscope into a cavity of the patient and administering at least some of the treatment locally using the endoscope (e.g., endoscope 52 shown in FIG. 3). Endoscopes may be rigid (e.g., a trocar) or flexible, and may include imaging, illumination, or operative features to assist the surgeon with the endoscopy. One example of an endoscope that may be incorporated into the electroporation system 10 is described in U.S. Pat. No. 6,181,964, hereby incorporated by reference herein in its entirety. With reference to FIG. 3, in some embodiments, endoscopes 52 also include a working channel 54 that extends from an upper or proximal end of the endoscope (e.g., a control section that is actuated by the user) to a distal end 56 of the endoscope through which one or more instruments, such as applicator 14, may be inserted to conduct the endoscopic procedure. In some instances, a flexible endoscope may have a narrower working channel than a rigid endoscope. As is known in the art, a flexible endoscope is typically used for procedures where the access pathway is via a conduit, such as in an esophageal approach to reach the lungs, while a rigid endoscope is typically used for procedures where the access pathway is a “line of sight” into the patient and to the particular tissue, such as is used in many abdominal procedures.

Endoscopic electroporation may involve inserting at least a portion of an applicator through the working channel of the endoscope to apply an electric field to the tissue adjacent to the distal end of the endoscope. In some examples, the slidable connection holding the applicator and the endoscope together may be controllable such that once the endoscope is advanced to a location in the body approaching the target site for the electroporation therapy, the applicator may be controllably advanced relative to the endoscope so that a distal end of the applicator reaches the target site while the endoscope remains at a distance relative to the target site. As discussed elsewhere herein, embodiments of the applicator may be mechanically steerable such that the tip may be steered to the target site via controls at or proximate the handle of the applicator. The control mechanism may be established based on direct visualization (e.g., a camera associated with the endoscope), surgical navigation, manual guidance based on the expected friction between the applicator surface and the interior surface of the endoscope, or other parameters as may be applicable for the particular structures included in the system. This controllable advancement of the applicator relative to the endoscope is of particular advantage where access to the target site involves passage through an internal vessel that is small in diameter. In such circumstances, the smaller diameter of the applicator relative to the endoscope allows the applicator to be advanced independently at lesser risk to the patient. This circumstance may arise, for example, where a tumor to be treated is in the cerebrum and intra-cranial blood vessels must be traversed to reach the tumor.

The electroporation system 10 can be used in any endoscopic access approach desired to fulfill its use and purpose. For example, in some embodiments, the electroporation system 10 may be used with an Olympus® EBUS Bronchoscope for performing bronchoscopy. In some embodiments, a flexible laparoscopic instrument may be used with the transformable needle 500 of the applicator disposed therein. Further, in some embodiments, the applicator may be inserted directly into a keyhole opening in the patient (e.g., with the laparoscopic device). In this arrangement, the keyhole opening in the body of the patient operates as the working channel during the electroporation procedure. Thus, in some examples, the system may include an applicator with an insertion end that is configured to be advanced to the target site unenclosed by an insertion device. In some examples, the properties and structure of the transformable needle 500 may be modified to accommodate use of the applicator as a standalone access element in the procedure. In the aforementioned examples, the system is complete without an endoscope, though it may be used with any type of endoscopic instrument desired.

In some examples, the electroporation system 10 may include an integral, “all-in-one” system having any combination of one or more of an endoscope, drug delivery channel or applicator, electroporation applicator, steering system, vision system, and/or imaging system (e.g., ultrasound). Embodiments of each of the foregoing components may include those discussed elsewhere herein. In such embodiments, the applicator (e.g., including electrodes and/or a drug delivery channel) may be any of the applicators 14, 110 disclosed herein. In some embodiments, the applicator may be a retractable portion of the all-in-one system.

With reference to FIGS. 4A and 4B, an example applicator 110 is shown having a transformable needle 500, an actuator 112, and a control portion 114. In some examples, the transformable needle 500 may have a diameter less than an internal diameter of the working channel of a cannulated access instrument, such as an endoscope (e.g., working channel 54 of endoscope 52 shown in FIG. 3), so that the transformable needle 500 may be inserted into the working channel and may extend from the control portion 114 to a position outside the endoscope at the external end (e.g., the end outside the patient) to the endoscopic site within the patient at the distal end of the endoscope. The transformable needle 500 may be longer than the working channel of the endoscope. The transformable needle 500 may also include one or more channels extending therethrough to allow the various components described herein to extend into the patient for treatment. For example, the actuator 112 may be movably engaged with at least a portion of the control portion 114 and a portion of the actuator may extend into the transformable needle 500 to allow a user to apply a force from a switch 116 to move the expandable portion 505, 510 into the deployed position as described herein. In some embodiments, the transformable needle 500 may act as the deployment mechanism discussed below. In some embodiments, a second switch 116 or other actuating device or structure may be used. The control portion 114 may include a body 120 and at least one end cap 122, which may support the transformable needle 500 therein. Referring now to FIG. 4B, in some embodiments, the actuator 112 may include a thumb switch 116 that is slidingly attached to the control portion 114 and engaged with a hollow mandrel 124 of the actuator via a connector 126 (e.g., a lure lock). The mandrel 124 may be directly or indirectly connected to the transformable needle, such that when the actuator 112 is slid forward on the control portion 114 by a user sliding switch 116, the switch 116 pushes the hollow mandrel 124 axially forward, which causes the entire transformable needle and/or the expandable portions 505, 510 to extend from the transformable needle 500 (e.g., either directly or indirectly as described herein). In this manner, the actuator 112, including the switch 116, mandrel 124, and pushing element 128, may extend at least partially into the transformable needle 500 to drive the expandable portions into the deployed position such that the expandable portions 505, 510 may act as electrodes. In various embodiments using drug delivery and electroporation in the same needle, the drug delivery channel 18 may be connected to the transformable needle 500 (e.g., disposed within the transformable needle 500).

### Electrode Deployment

Referring now to FIGS. 5A and 5B, an example embodiment of the transformable needle is shown with the deployment mechanism (e.g., actuator 112) to be used in an applicator, such as the applicator shown in FIGS. 4A and 4B. In various embodiments, the deployment mechanism 112 may be disposed within a control portion, such as the control portion 114 shown in FIG. 4A. In various embodiments, a deployment mechanism is in operably communication with the proximal end of the first expandable portion and the second expandable portion. In various embodiments, the deployment mechanism may be configured to allow the first expandable portion and the second expandable portion to move between the retracted position and the deployed position. For example, the deployment mechanism may be the hollow mandrel 124 discussed in reference to FIGS. 4A-4B. In various embodiments, the transformable needle 500 may include a deployment mechanism switch 116 configured to move the deployment mechanism between the retracted position and the deployed position, such as by applying one or more forces axially along the needle.

Referring now to FIG. 5B, in various embodiments, the transformable needle 500 may include a first electroporation member 501, a second electroporation member 502, and a needle casing 545. In various embodiments, the transformable needle 500 may also define a drug delivery channel 18. In some embodiments, a needle tip 571 may be provided (e.g., either attached to or integral with the needle casing 545). The first electroporation member 501 may define a first expandable portion 505 and the second electroporation member 502 may define a second expandable portion 510. At least a portion of the first expandable portion 505 and second expandable portion 510 of FIG. 5 may correspond to the electrodes 100 discussed above, whereby an electric field may be created between at least a portion of each of the first expandable portion and the second expandable portion. The expandable portions 505, 510 may initially be parallel and adjacent to each other to define a diameter substantially comparable to a single needle (e.g., the diameter of the needle casing 545), and when deployed, the expandable portions may extend laterally outwardly relative to the longitudinal axis of the needle to create a gap between a portion of the expandable portions and the rest of the needle assembly. In various embodiments, the first electroporation member 501 and the second electroporation member 502 may be configured to move between a retracted position and a deployed position to facilitate insertion into the patient and electroporation respectively. Additional portions of the electroporation members may be disposed distal (e.g., closer to the tip 571) of the respective expandable portion and proximal (e.g., closer to the actuator 116) of the respective expandable portions as shown in FIG. 5B. At least the proximal portions may be actuated by the actuator to cause the proximal end of the first and second portions to move upwardly relative to the distal ends of the first and second portions to cause the portions to expand outwardly. While the disclosure uses two expandable portions to describe the inventions, additional expandable portions may be used in accordance with the present disclosure. For example, in various embodiments, the transformable needle may have one or more additional pairs of expandable members at a plurality of radial positions around the outer casing. The transformable needle 500 may have 2, 4, 6, 8, or more expandable electrodes associated with the single needle, with each respective pair of expandable portions lying on a different plane that intersects the central, longitudinal axis of the needle casing. For example, a two-electrode embodiment described herein depicts a pair of electrodes lying on a plane that intersects the central, longitudinal axis of the insertion tube, which electrodes are disposed 180 degrees from each other relative to the central, longitudinal axis. A four-electrode embodiment may, in various embodiments, include the two electrodes described above and a second set of identical electrodes lying on a plane perpendicular to the plane of the first pair of electrodes also intersecting the central, longitudinal axis. Additional embodiments may be envisioned at any respective radial position relative to the central, longitudinal axis.

In various embodiments, the first electroporation member 501 may define a proximal end 503 and a distal end 504. In various embodiments, the first electroporation member 501 may define the first expandable portion 505. In various embodiments, the first expandable portion 505 may be defined between the proximal end 503 and the distal end 504. In various embodiments, the distal end of the first expandable portion 505 may be the same point as the distal end 504 of the first electroporation member 501, such that the first expandable portion 505 is defined at approximately the distal end 504 of the first electroporation member 501. In various embodiments, the first expandable portion 505 may define a first conductive portion 515 and a first non-conductive portion 525. In various embodiments, the first expandable portion 505 may be made, at least partially, out of a material with shape memory characteristics, such as nitinol (nickel-titanium). In various embodiments, the first conductive portion 515 may have a conductive material and/or coating. For example, the first conductive portion 515 may have a gold coating, which in some examples may minimize electrical noise and/or prevent shedding of unwanted materials of the first expandable portion 505 (e.g., nickel) into a patient's body. In various embodiments, the first non-conductive portion 525 may be coated in a non-conductive material, such as a non-conductive coating that may, for example, be disposed over a common conductive material between the conductive and non-conductive portions (e.g., as shown by the shading in FIG. 5B).

As shown, in some embodiments, the first expandable portion 505 may have a first transition point 535 configured between the first conductive portion 515 and the first non-conductive portion 525. In some embodiments, the first transition point 535 may be at or near the middle of the first expandable portion 505. In various embodiments, as the first expandable portion 505 moves from the retracted position to the deployed position, the first transition point 535 may move away from the needle casing 545. In some embodiments, the first transition point 535 may be a hinge. Alternatively, such as, but not limited to, in an instance the first electroporation member is made out of a material with shape memory characteristics (e.g., nitinol), the first transition point 535 may be an area approximately between the first conductive portion 515 and the first non-conductive portion 525 that configured to expand outward from the needle casing 545 in an instance in which the first electroporation member 501 is moved from the retracted position into the deployed position (e.g., the first transition point 535 may be a rounded or curved area of the first expandable portion 505 when in the deployed position, while the first transition point 535 may be held approximately straight when in the retracted position).

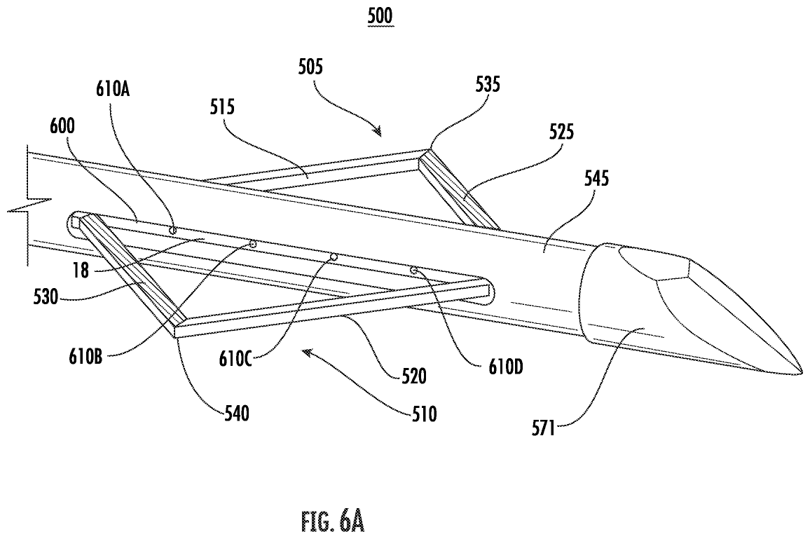

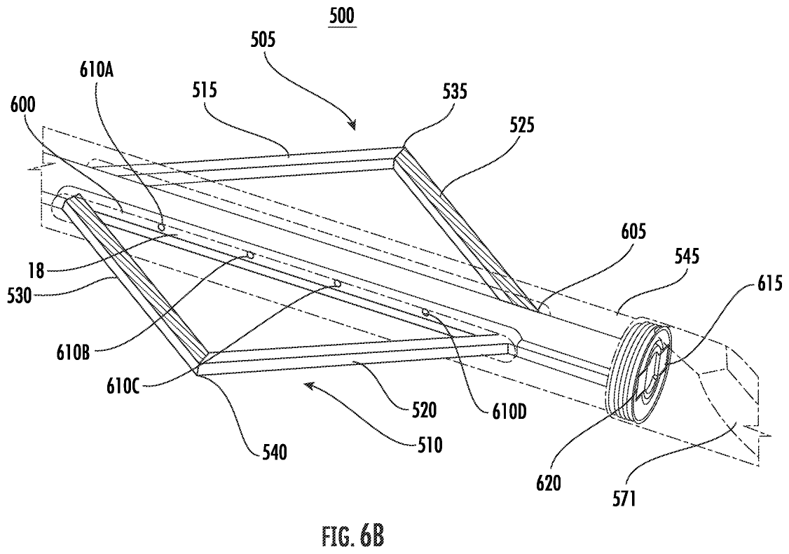

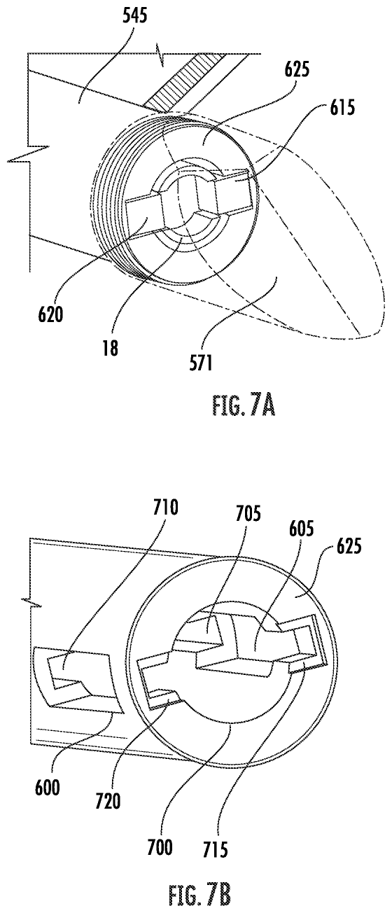

As discussed below in reference to FIGS. 7A and 7B, the first electroporation member 501 may be at least partially received by a first electroporation member channel 705 defined in the casing 545. In various embodiments, as shown in FIGS. 6B and 7A, the first electroporation member 501 may define a first attachment mechanism 615. In various embodiments, the first attachment mechanism 615 may be defined at approximately the distal end 604 of the first electroporation member 501. In various embodiments, the first attachment mechanism 615 may be configured to be received by a first attachment mechanism receiver 715 (shown in FIG. 7B) defined by the needle casing 545. In various embodiments, the first attachment mechanism 615 may be configured to restrict the movement of the first electroporation member 501 relative to the distal end 625 of the needle casing. For example, the distal end 504 of the first electroporation member 501 may be fixed (e.g., by the first attachment mechanism 615) to the needle casing 545 in an instance in which the proximal end of the first electroporation member 501 is not fixed to the needle casing 545 and is operable by the actuator 112. In various embodiments, in an instance in which the first electroporation member 501 is moving from the deployed position into the retracted position, the proximal end moves away from the distal end 501, which remains fixed relative to the casing. In various embodiments, in an instance in which the first electroporation member 501 is moving from the retracted position into the deployed position, the proximal end moves towards the distal end 501, which remains fixed relative to the casing. In various embodiments, the second electroporation member 502 may define a proximal end 508 and a distal end 509. In various embodiments, the second electroporation member 502 may be configured the same as the first electroporation member 501. In various embodiments, the second electroporation member 502 may define a second expandable portion 510. In various embodiments, the second expandable portion 510 may be defined between the proximal end 508 and the distal end 509. In various embodiments, the distal end of the second expandable portion 510 may be the same point as the distal end 509 of the second electroporation member 502, such that the second expandable portion 510 is defined at approximately the distal end 509 of the second electroporation member 502. In various embodiments, the second expandable portion 510 may define a second conductive portion 520 and a second non-conductive portion 530. In various embodiments, the second expandable portion 510 may be made, at least partially, out of a material with shape memory characteristics, such as nitinol (nickel-titanium). In various embodiments, the second conductive portion 520 may be a conductive material and/or coating. For example, the second conductive portion 520 may have a gold coating, which in some examples may minimize electrical noise and/or prevent shedding of unwanted materials of the second expandable portion 510 (e.g., nickel) into a patient's body. In various embodiments, the second non-conductive portion 530 may be coated in a non-conductive material, such as a non-conductive coating that may, for example, be disposed over a common conductive material between the conductive and non-conductive portions (e.g., as shown by the shading in FIG. 5B).

As shown, in some embodiments, the second expandable portion 510 may have a second transition point 540 configured between the second conductive portion 520 and the second non-conductive portion 530. In various embodiments, the second transition portion may be configured the same as the first transition point 535. In some embodiments, the second transition point 540 may be at or near the middle of the second expandable portion 510. In various embodiments, as the second expandable portion 510 moves from the retracted position to the deployed position, the second transition point 540 may move away from the needle casing 545. In some embodiments, the second transition point 540 may be a hinge. Alternatively, such as, but not limited to, in an instance the second electroporation member 502 is made out of a material with shape memory characteristics (e.g., nitinol), the second transition point 540 may be an area approximately between the second conductive portion 520 and the second non-conductive portion 530 that configured to expand outward from the needle casing 545 in an instance in which the second electroporation member 502 is moved from the retracted position into the deployed position (e.g., the second transition point 540 may be a rounded or curved area of the second expandable portion 510 when in the deployed position, while the second transition point 540 may be held approximately straight when in the retracted position).

As discussed below in reference to FIGS. 7A and 7B, the second electroporation member 502 may be at least partially received by a second electroporation member channel 710 defined in the casing 545. In various embodiments, as shown in FIGS. 6B and 7A, the second electroporation member 502 may define a second attachment mechanism 620. In various embodiments, the second attachment mechanism 620 may be defined at approximately the distal end 608 of the second electroporation member 502. In various embodiments, the second attachment mechanism 620 may be configured to be received by a second attachment mechanism receiver 720 (shown in FIG. 7B) defined on the needle casing 545. In various embodiments, the second attachment mechanism 620 may be configured to restrict the movement of the second electroporation member 502 relative to the distal end 625 of the needle casing. For example, the distal end 509 of the second electroporation member 502 may be fixed (e.g., by the second attachment mechanism 620) to the needle casing 545 in an instance in which the proximal end of the second electroporation member 502 is not fixed to the needle casing 545 and is operable by the actuator 112. In various embodiments, in an instance in which the second electroporation member 502 is moving from the deployed position into the retracted position, the proximal end moves away from the distal end 509, which remains fixed relative to the casing. In various embodiments, in an instance in which the second electroporation member 502 is moving from the retracted position into the deployed position, the proximal end moves towards the distal end 509, which remains fixed relative to the casing.

As shown, in the deployed position, the first expandable portion 505 and the second expandable portion 510 may define a generally diamond shape. In some embodiments, the first conductive portion 515 of the first expandable portion 505 and the second conductive portion 520 of the second expandable portion 510 may be parallel or approximately parallel in the deployed position. Additionally, in some embodiments, the first non-conductive portion 525 of the first expandable portion 505 and the second non-conductive portion 530 of the second expandable portion 510 may be parallel or approximately parallel in the deployed position. In some embodiments, in the deployed position, a needle casing 545 may be positioned between the first expandable portion 505 and the second expandable portion 510. In some embodiments, one or both of the expandable portions 505, 510 may have sharp edges along some or all of their length that are configured to cut tissue during operation.

In various embodiments, in the deployed position, the needle casing 545 and/or the drug delivery channel 18 may remain between the first expandable portion 505 and the second expandable portion 510 in an instance in which the first expandable portion 505 and the second expandable portion 510 are in the deployed position. In various embodiments, in the deployed position, the first conductive portion 515 of the first expandable portion 505 and the second conductive portion 520 of the second expandable portion 510 may be electrically isolated from each other in the transformable needle 500, such that application of a voltage between the first conductive portion 515 and the second conductive portion 520 may create an electric field between them in at least a portion of the target tissue during use. In various embodiments, the first conductive portion 515 and the second conductive portion 520 may be diagonally positioned from one another in an instance in which the transformable needle 500 is in the deployed position. For example, as shown in FIG. 5B, the first conductive portion 515 and the second conductive portion 520 are positioned diagonally from one another in the deployed position, with the respective conductive portions lying parallel to each other. While FIG. 5B illustrates the first conductive portion 515 closer towards the proximal end 503 of the first electroporation member 501 than the first non-conductive portion 525 and the second conductive portion 520 closer towards the distal end 509 of the second electroporation member 502 than the second non-conductive portion 530, in various embodiments, the first conductive portion 515 may be closer towards the distal end 504 of the first electroporation member 501 than the first non-conductive portion 525 and the second conductive portion 520 may be closer towards the proximal end 508 of the second electroporation member 502 than the second non-conductive portion 530 (e.g., a diagonal relationship between the first conductive portion 515 and the second conductive portion 525 may be present).

In various embodiments, as discussed herein, the transformable needle 500 (e.g., the first expandable portion 505 of the first electroporation member 501 and the second expandable portion 510 of the second electroporation member 502) may be configured to move between the deployed position (e.g., FIGS. 5A and 5B) and a retracted position. In various embodiments, the retracted position may be the position of the first expandable portion 505 and the second expandable portion 510 in an instance the transformable needle is being inserted or removed from a patient (e.g., through a hole smaller than the diamond shape of the expandable portions in the deployed position). In various embodiments, the distance between the first conductive portion 515 of the first expandable portion 505 and the second conductive portion 520 of the second expandable portion 510 may be less in the retracted position than in the deployed position. In the retracted position, the angle defined between the conductive portion and the non-conductive portion of each expandable portion may be approximately 180 degrees. For example, the angle defined at the first transition point 535 of the first expandable portion 505 between the first conductive portion 515 and the first non-conductive portion 525 may be approximately 180 degrees. In various embodiments, the first expandable portion 505 and the second expandable portion 510 may be generally parallel with the needle body 560 in the retracted position.

FIGS. 6A and 6B show a closer view of the distal end of the transformable needle 500 with the first expandable portion 505 and the second expandable portion 510 in the deployed position. FIG. 6A is an exterior view of the transformable needle 500 and FIG. 6B illustrates an interior view of the transformable needle 500 inside the casing 545. In various embodiments, as discussed in more detail in reference to FIGS. 7A and 7B below, the needle casing 545 may define a first electroporation aperture 605 and a second electroporation aperture 600. In various embodiments, the first electroporation aperture 605 and the second electroporation aperture 600 may define a length at least approximately as long as the respective deployed expandable portion. In various embodiments, the first electroporation aperture 605 and the second electroporation aperture 600 may define a length equal to or slightly greater than an axial length of the respective deployed expandable portion. The length of the apertures 605, 600 may be configured to allow the expandable portions to deploy while not creating unnecessary gaps.

In various embodiments, the length of the first electroporation aperture 605 and the second electroporation aperture 600 and the length of the expandable portions may be based on the particular application of the transformable needle. In various embodiments, the length of the first electroporation aperture 605 and the second electroporation aperture 600 may be based on the desired size of the treatment area (e.g., the length may be longer to treat larger tumors with larger conductive portions of the electrodes).

In various embodiments, the transformable needle may be dimensioned for one or more particular target tissues and methods, as would be understood by a person of ordinary skill in the art in light of the present disclosure. In various embodiments, the transformable needle may be used for, for example, intravenous delivery, bronchial delivery, subcutaneous delivery (e.g., treating breast tumors), brain delivery (e.g., inserted via the nasal cavity), gastrointestinal delivery, and/or any other possible treatment location depending upon the target tissue. In some examples, the first electroporation aperture 605 and the second electroporation aperture 600 may define a length of at least 4.5 millimeters. In some embodiments, the electrodes may be positioned approximately 2 millimeters to approximately 4 millimeters from one another diagonally in the deployed position in some examples (e.g., for intravenous delivery). In some embodiments, the electrodes may be positioned approximately 1 centimeter from one another diagonally in the deployed position in some examples (e.g., for subcutaneous delivery).

In various embodiments, the first expandable portion 505 of the first electroporation member 501 and the second expandable portion 510 of the second electroporation member 502 may be configured to expand through the respective electroporation aperture 605, 600. For example, the first electroporation member 501 and the second electroporation 502 may each be coupled to the needle casing 545 on one side of the respective expandable portion 505, 510 (e.g., the first attachment mechanism 615 of the first electroporation member 501 and the second attachment mechanism 620 of the second electroporation member 502 may each be attached to the needle casing at approximately the distal end 625 of the needle casing such that the distal ends of the expandable portions do not move relative to the casing). In various embodiments, the other, proximal side of the respective expandable portions 505, 510 may be operably coupled with the deployment mechanism 112 and not affixed to the needle casing 545, such that the movement of the deployment mechanism may cause the electroporation members 501, 502 to move within the respective electroporation member channel 705, 710 (shown in FIG. 7B), such that the respective expandable portions 505, 510 may move between the retracted position and the deployed position. For example, the deployment mechanism may be the hollow mandrel 124 discussed in reference to FIGS. 4A-4B, or any other physical connection between the electroporation members 501, 502 and the actuator 112. In various embodiments, the transformable needle 500 may include a deployment mechanism switch 116 configured to move the deployment mechanism between the retracted position and the deployed position. For example, the deployment mechanism switch 116 may be an actuator (e.g., the actuator 112 of FIG. 4A) used to move the first expandable portion 505 and the second expandable portion 510 between the retracted position and the deployed position.

In various embodiments, the transformable needle 500 may also include one or more drug delivery channels 18 for transmitting one or more treatment agents to the treatment site. In various embodiments, the drug delivery channel 18 may be disposed within the needle casing 545. In some embodiments, the drug delivery channel 18 may be affixed to the needle casing 545 and/or other components of the applicator. As discussed above, the drug delivery channel 18 may be in communication with the drug delivery device 16 to receive one or more treatment agents, such that the one or more treatment agents may be provided through the injection ports in an instance in which the transformable needle 500 is in the deployed position. In various embodiments, the drug delivery device 16 may provide the treatment agent(s) either manually (e.g., using a syringe) or automated (e.g., using a robotic system). In some examples, a method of treating a lesion (e.g., cancer cells) may be performed with the aid of robotics. For instance, the applicator may be used with a robotic system to perform the procedure. In particular, the applicator may be advanced through the body of the patient and/or the electrodes of the applicator may be deployed through control of the robotic device of the robotic system. To perform these functions, for example, an arm of the robotic device may be manipulated to rotate and position the applicator during the procedure. Similarly, the arm of the robotic device may be manipulated to control electricity flow into the applicator. In some examples, other steps of the method may also be aided by the use of the robotic system.

In various embodiments, the drug delivery channel 18 may be fixed relative to the needle casing 545. In various embodiments, the drug delivery channel 18 may define one or more delivery side ports (e.g., delivery side ports 610A-610D) configured to release one or more treatment agents laterally from the drug delivery channel. In various embodiments, the delivery side ports 610 may be aligned with one of the electroporation apertures 600, 605 (e.g., the delivery side ports 610A-D shown aligned with the second electroporation aperture 600). In various embodiments, in the retracted position, the first expandable portion 505 of the first electroporation member 501 and the second expandable portion 510 of the second electroporation member 502 may at least partially cover the delivery side ports 610, such that the treatment agent may not be dispersed to the treatment site until the expandable portions 505, 510 are moved to the deployed position. In some embodiments, the delivery side ports 610 may be along the drug delivery channel 18 within the diamond defined between the first expandable portion 505 and the second expandable portion 510 in the deployed position, such that the electric field generated by the conductive portions of the expandable portions acts on the treatment agent.

FIGS. 7A and 7B show the distal end 625 of the needle casing 545 in operation (FIG. 7A) and with the needle casing alone (FIG. 7B). As shown in FIG. 7B, the needle casing 545 may define a drug delivery aperture 700, a first electroporation member channel 705, and a second electroporation member channel 710. In various embodiments, as shown in FIG. 7A, the distal end 625 of the needle casing 545 may be configured to operably couple with a needle tip 571. Alternatively, the needle tip 571 may be integral to the needle casing 545. In various embodiments, the needle tip 571 may be a closed point configured to allowed the transformable needle to more easily penetrate tissue during operation insertion and/or removal of the transformable needle from a patient. In various embodiments, the needle casing 545 may comprise a non-conductive material, such as Polyether ether ketone (PEEK). In various embodiments, the needle casing 545 may include an attachment mechanism for the needle tip. For example, the needle casing 545 may have a threading at the distal end 625 configured to receive the needle tip 571. In various embodiments, the needle tip 571 may be a sharp metallic end configured to, in some examples, pierce the tissue and/or tumor of a patient. In various embodiments, the needle tip 517 may have a threaded stainless-steel point configured to screw onto the distal end 625 of the needle casing 545, which may be non-conductive as discussed above. In some embodiments, the electroporation members 501, 502 may be electrically isolated from each other, such that the casing 545, drug delivery channel 18, and/or any additional components within the applicator are non-conductive or the electroporation members are selectively insulated to prevent shorting between the electroporation members.

In various embodiments, the first electroporation member channel 705 may be configured to receive the first electroporation member 501. In various embodiments, the first electroporation member channel 705 may include a first electroporation aperture 605. In various embodiments, the first expandable portion 505 may be configured to align with the first electroporation aperture 605 at least in an instance in which the first electroporation member 501 is in the deployed position. In various embodiments, the first electroporation member channel 705 may include a first attachment mechanism receiver 715 configured to receive the first attachment mechanism 615 of the first electroporation member 501. In various embodiments, the first attachment mechanism receiver 715 may be disposed at approximately the distal end 625 of the needle casing 545. In operation, the first attachment mechanism 615 may engage the first attachment mechanism receiver 715 laterally from a radially-inward direction, such that the attachment mechanism receiver prevents the attachment mechanism from moving axially and is removable by depressing the attachment mechanism towards an axial center of the casing.

In various embodiments, the second electroporation member channel 710 may be configured to receive the second electroporation member 502. In various embodiments, the second electroporation member channel 710 may include a second electroporation aperture 600. In various embodiments, the second expandable portion 510 may be configured to align with the second electroporation aperture 600 at least in an instance in which the second electroporation member 502 is in the deployed position. In various embodiments, the second electroporation member channel 710 may include a second attachment mechanism receiver 720 configured to receive the second attachment mechanism 620 of the second electroporation member 502. In various embodiments, the second attachment mechanism receiver 720 may be disposed at approximately the distal end 625 of the needle casing 545. In operation, the second attachment mechanism 620 may engage the second attachment mechanism receiver 720 laterally from a radially-inward direction, such that the attachment mechanism receiver prevents the attachment mechanism from moving axially and is removable by depressing the attachment mechanism towards an axial center of the casing.

In various embodiments, the drug delivery aperture 700 may be configured to receive the drug delivery channel 18. In various embodiments, the drug delivery channel 18 may be fixed relative to the needle casing 545, such that the delivery side ports 610 are aligned with one of the first electroporation aperture 605 and/or the second electroporation aperture 600. In various embodiments, as shown in FIG. 7A, the drug delivery channel 18 may be configured to receive the first attachment mechanism 615 and/or the second attachment mechanism, such that the distal end 504, 509 of the respective electroporation member 501, 502 may be fixed relative to the drug delivery channel 18. The drug delivery channel 18 may receive treatment agent from the applicator and/or via a separate supply device using any means, including those described herein.

Nitinol is a shape memory alloy capable of “remembering” a programmed shape and returning to the programmed shape under certain temperature conditions. Nitinol may be programmed to a specific shape (e.g., deployed position of the expandable portions) by holding the nitinol in a predetermined position and heating the nitinol to about 500° C. (approximately 932° F.) to set the shape of the nitinol. After shape setting, the nitinol may be cooled to room temperature and mechanically deformed into a second shape (e.g., retracted position of the expandable portions). During use, when the nitinol is heated above a transformation temperature (e.g., body temperature or below body temperature but above ambient temperature), the nitinol returns to its programmed shape. By adjusting the proportions of nickel and titanium in the Nitinol, the transformation temperature of the nitinol may be tuned relative to human body temperature, such that the Nitinol changes shape upon coming into contact with the temperature of the patient's body tissue. In use, nitinol may have a “start” temperature and a “finish” temperature at which the transformation begins and ends, respectively. In some embodiments, the finish temperature may be less than or equal to body temperature. For example, in some embodiments, the nitinol may include 54.5% nickel and 45.5% titanium, which may have a transformation temperature of 60° Celsius. In some embodiments, the transition temperature of the Nitinol may be human body temperature. Alternatively, rather than relying on the body temperature of the patient to warm the Nitinol, the expandable portions 505, 510 may instead change shape upon a voltage passing through it, whether it be the actual voltage being used for electroporation, or some amount of pre-voltage, such as a smaller voltage with a sole intended use of assisting the electrodes to change shape. Once the shape has been changed, the standard voltage may be passed through the electrodes.

### Example Use

In an example use case, the transformable needle 500 may be positioned in the retracted position within the transformable needle 500 in an instance the transformable needle is inserted into a body. During operation, the transformable needle 500 may be positioned near the target (e.g., a tumor), as which point the needle may be extended out of the transformable needle 500, as discussed herein, to engage with the target. In various embodiments, the needle tip 571 may then engage with the target (e.g., a tumor) to puncture said target. In an instance the transformable needle 50 is within the target, the first expandable portion 505 and the second expandable portion 510 may be moved from the retracted position to the deployed position (e.g., as shown in FIG. 5) via the deployment mechanism. Once in the deployed position, the first conductive portion 515 of the first expandable portion 505 and the second conductive portion 520 of the second expandable portion 510 are electrically isolated from each other in the transformable needle, such that application of a voltage (e.g., via the generator 12) between the first conductive portion 515 and the second conductive portion 520 is configured to create an electric field in at least a portion of the target tissue. Additionally, the chosen drug may be delivered to the target via the delivery side ports 610 of the drug delivery channel 18. After the electroporation has been completed, the transformable needle 500 may be moved from the deployed position to the retracted position (e.g., via activating the deployment mechanism switch) to remove the transformable needle from the body. In some embodiments, the transformable needle 500 may be moved to the retracted position by pulling the expandable portions 505, 510 back into the transformable needle 500 discussed herein. Alternatively, the transformable needle 500 may be removed from the body while still in the deployed position (e.g., the insertion point of the body may be large enough to allow the needle to be removed).

### Example Electrical Parameters

In various embodiments, in the deployed position, the first conductive portion 515 of the first expandable portion 505 and the second conductive portion 520 of the second expandable portion 510 may be electrically isolated from each other in the transformable needle, such that application of a voltage between the first conductive portion and the second conductive portion may create an electric field in at least a portion of the target tissue. The nature of the electric field to be generated by the generator 12 is determined by the nature of the tissue, the size of the selected tissue and its location. It is desirable that the field be as homogenous as possible and of the correct amplitude. Excessive field strength results in lysing of cells, whereas a low field strength results in reduced efficacy.

In the depicted embodiments, the nominal electric field can be designated either “high” or “low”. The following paragraphs describe electrical parameters for system including a high voltage generator followed by a system including a low voltage generator.

Turning to high voltage systems specifically, i.e., those having a high electric field, in some embodiments, it is preferable that the nominal electric field is from about 700 V/cm to 1500 V/cm. In some embodiments, it is further preferable that the nominal electric field is from about 1000 V/cm to 1500 V/cm. In some embodiments, the high electric field may be about 1500 V/cm. With regard to pulse duration for high voltage systems, in some embodiments, a pulse duration of less than 1 ms may be used. In some embodiments, a pulse duration between 100 s and 1 ms may be used.

Turning to low voltage systems specifically, in some embodiments, the generator may be a low-voltage generator. The electroporation therapy may be administered using the low-voltage generator producing an electric field of 700 V/cm or less, 600 V/cm or less, 500 V/cm or less, 400V/cm or less, 300V/cm or less, 200V/cm or less, or 100V/cm or less. The electroporation therapy may be administered using the low-voltage generator producing an electric field from 700 V/cm to 10 V/cm. The electroporation therapy may be administered using the low-voltage generator producing an electric field from 600 V/cm to 10 V/cm. The electroporation therapy may be administered using the low-voltage generator producing an electric field from 500 V/cm to 10 V/cm. The electroporation therapy may be administered using the low-voltage generator producing an electric field from 400 V/cm to 10 V/cm. The electroporation therapy may be administered using the low-voltage generator producing an electric field from 300 V/cm to 10 V/cm. The electroporation therapy may be administered using the low-voltage generator producing an electric field from 700 V/cm to 60 V/cm. The electroporation therapy may be administered using the low-voltage generator producing an electric field from 600 V/cm to 60 V/cm. The electroporation therapy may be administered using the low-voltage generator producing an electric field from 500 V/cm to 60 V/cm. The electroporation therapy may be administered using the low-voltage generator producing an electric field from 400 V/cm to 60 V/cm. The electroporation therapy may be administered using the low-voltage generator producing an electric field from 300 V/cm to 60 V/cm. The electroporation therapy may be administered using the low-voltage generator producing an electric field from 700 V/cm to 100 V/cm. The electroporation therapy may be administered using the low-voltage generator producing an electric field from 600 V/cm to 100 V/cm. The electroporation therapy may be administered using the low-voltage generator producing an electric field from 500 V/cm to 100 V/cm. The electroporation therapy may be administered using the low-voltage generator producing an electric field from 400 V/cm to 100 V/cm. The electroporation therapy may be administered using the low-voltage generator producing an electric field from 300 V/cm to 100 V/cm. The electroporation therapy may be administered using the low-voltage generator producing an electric field from 300 V/cm to 200 V/cm. The electroporation therapy may be administered using the low-voltage generator producing an electric field from 400 V/cm to 300 V/cm. In some embodiments, the pulse duration of the low-voltage generator may be from 1 millisecond (ms) to 1 second (s).

Preferably, when low fields are used, the nominal electric field is from about 10 V/cm to 400 V/cm. In some embodiments, the nominal electric field may be from about 25 V/cm to 75 V/cm. In some embodiments, the low nominal electric field may be about 400 V/cm. In a particular embodiment, it is preferred that when the electric field is low, the pulse length is long relative to a high field pulse. For example, when the nominal electric field is in the “low” range discussed herein, it is preferred that the pulse length is about 10 msec.

In some embodiments, the electroporation therapy comprises the administration of one or more voltage pulses having a duration of approximately 0.1 ms each. The voltage pulse that can be delivered to the tumor may be about 400V/cm for low-voltage generators and 1500V/cm for high-voltage generators. In another embodiment, the checkpoint inhibitor is administered systemically. In some embodiments, either a high or a low voltage may be used with the treatment therapies and apparatus disclosed herein.

### Methods of Treatment

The electroporation devices described herein may be used in therapeutic treatments and in the delivery of treatment agents. In some embodiments, therapeutic treatments include electrotherapy, also referred to herein as electroporation therapy (EPT), using the described apparatuses for the delivery of one or more treatment agents (e.g., molecules) to a cell, group of cells, or tissue and for performing electroporation on the cell, group of cells, or tissue. In some embodiments, the molecule or treatment agent is a drug (i.e., active pharmaceutical ingredient). Combining any of the treatment agent(s) discussed herein or otherwise generally known in the art with EPT, as discussed herein, may provide an effective treatment even in patients who did not respond to the treatment agent(s) on their own. In some embodiments, the drug is a small molecule. In some embodiments, the drug is a macromolecule. A drug can be, but is not limited to, a chemotherapeutic agent. A macromolecule can be, but is not limited to, a chemotherapeutic agent, nucleic acid (such as, but not limited to, polynucleotide, oligonucleotide, DNA, cDNA, RNA, peptide nucleic acid, antisense oligonucleotides, siRNA, miRNA, ribozyme, plasmid, and expression vector), and polypeptide (such as, but not limited to, peptide, antibody, and protein). In some embodiments, therapeutic treatments include delivery of a therapeutic electric pulse to a cell, group of cells, or tissue using any of the described electroporation devices. The cell, group of cells, or tissue may be, but is not limited to, a tumor cell or tumor tissue.

Drugs or treatment agents contemplated for use with the methods include chemotherapeutic agents having an antitumor or cytotoxic effect. A drug can be an exogenous agent or an endogenous agent. In some embodiments, the drug is a small molecule exogenous agent. Small molecule exogenous agent agents include, but are not limited to, bleomycin, neocarcinostatin, suramin, doxorubicin, carboplatin, taxol, mitomycin C and cisplatin. Other chemotherapeutic agents will be known to those of skill in the art (see, for example, The Merck Index). In some embodiments, the drug is a membrane-acting agents. “Membrane acting” agents act primarily by damaging the cell membrane. Non-limiting examples of membrane-acting agents include, N-alkylmelamide and para-chloro mercury benzoate. In some embodiments, the drug is a cytokine, chemokine, lymphokine, or hormone. In some embodiments, the drug is a nucleic acid. In some embodiments, the nucleic acid encodes one or more cytokines, chemokines, lymphokines, therapeutic polypeptide, adjuvant, or a combination thereof.

The molecule or treatment agent can be administered to a subject before, during, or after administration of the electric pulse. The molecule can be administered at or near the cell, group of cells or tissue in a patient. In some embodiments, the molecule can be co-localized with the electric pulse using an applicator having electrodes and a drug delivery channel extending therethrough as described herein. The chemical composition of the treatment agent will dictate the most appropriate time to administer the agent in relation to the administration of the electric pulse. For example, while not wanting to be bound by a particular theory, it is believed that a drug having a low isoelectric point (e.g., neocarcinostatin, IEP=3.78), would likely be more effective if administered post-electroporation in order to avoid electrostatic interaction of the highly charged drug within the field. Further, such drugs as bleomycin, which have a very negative log P, (P being the partition coefficient between octanol and water), are very large in size (MW=1400), and are hydrophilic, thereby associating closely with the lipid membrane, diffuse very slowly into a tumor cell and are typically administered prior to or substantially simultaneous with the electric pulse. In addition, certain treatment agents may require modification in order to allow more efficient entry into the cell. For example, an agent such as taxol can be modified to increase solubility in water which would allow more efficient entry into the cell. In some embodiments, electroporation facilitates entry of the molecule into a cell by creating pores in the cell membrane.

In some embodiments, the molecule or treatment agent is delivered to modulate expression of a gene. The term “modulate” envisions the decrease (suppression) or increase (stimulation) of expression of a gene. Where a cell proliferative disorder is associated with the expression of a gene, nucleic acid sequences that interfere with the gene's expression at the translational level can be used. In some embodiments, one or more antisense nucleic acids, ribozymes, siRNAs, miRNA, triplex agents, or the like are delivered via electroporation to block transcription or translation of a specific mRNA. In some embodiments, a nucleic acid is delivered to express an RNA or polypeptide. The nucleic acid can be recombinant, single stranded or double stranded, DNA or RNA or a combination of DNA and RNA, circular or linear, and/or supercoiled or relaxed. The nucleic acid can also be associated with one or more of proteins, lipids, virus, viral vector, chimeric virus, or viral particle. The nucleic acid can also be naked. A virus can be, but is not limited, adenovirus, herpes virus, vaccinia, DNA virus, RNA virus, retrovirus, murine retrovirus, avian retrovirus, Moloney murine leukemia virus (MoMuLV), Harvey murine sarcoma virus (HaMuSV), murine mammary tumor virus (MuMTV), Rous Sarcoma Virus (RSV), gibbon ape leukemia virus (GaLV) can be utilized. Similarly a viral vector, chimeric virus, and/or viral particle can be derived from any of the above described viruses.

### Therapeutic Polypeptides

Therapeutic polypeptides (one type of treatment agent listed above) include, but are not limited to, immunomodulatory agents, biological response modifiers, co-stimulatory molecule, metabolic enzymes and proteins, antibodies, checkpoint inhibitors, and adjuvants.

The term “immunomodulatory agents” is meant to encompass substances which are involved in modifying an immune response. Examples of immune response modifiers include, but are not limited to, cytokines, chemokines, lymphokines, and antigen binding polypeptides. Lymphokines can be, but not limited to, tumor necrosis factor, interleukins (IL, such as, but not limited to IL-1, IL-2, IL-3, IL-12, IL-15), lymphotoxin, macrophage activating factor, migration inhibition factor, colony stimulating factor, and alpha-interferon, beta-interferon, gamma-interferon, and their subtypes. In some embodiments, the immune response modifier comprises a nucleic acid encoding one or more cytokines, chemokines, lymphokines or subunits of cytokines, chemokines, and lymphokines. In some embodiments, the immunomodulatory agent is an immune stimulator. Non-limiting examples of immune stimulators include, IL-33, flagellin, IL-10 receptor, sting receptor, IRF3. The term “cytokine” is used as a generic name for a diverse group of soluble proteins and peptides which act as humoral regulators at nano- to picomolar concentrations and which, either under normal or pathological conditions, modulate the functional activities of individual cells and tissues. As used herein an “immunostimulatory cytokine” includes cytokines that mediate or enhance the immune response to a foreign antigen, including viral, bacterial, or tumor antigens. Immunostimulatory cytokines include, but are not limited to, TNFα, IL-1, IL-10, IL-12, IL-12 p35, IL-12 p40, IL-15, IL-15Rα, IL-23, IL-27, IFNα, IFNβ, IFNγ, IL-2, IL-4, IL-5, IL-7, IL-9, IL-21, and TGFβ. In some embodiments, the immunostimulatory cytokine is a nucleic acid encoding one or more of TNFα, IL-1, IL-10, IL-12, IL-12 p35, IL-12 p40, IL-15, IL-15Rα, IL-23, IL-27, IFNα, IFNβ, IFNγ, IL-2, IL-4, IL-5, IL-7, IL-9, IL-21, and TGFβ.

Another treatment agent, a “co-stimulator,” refers to any of a group of immune cell surface receptor/ligands which engage between T cells and antigen presenting cells and generate a stimulatory signal in T cells which combines with the stimulatory signal (i.e., “co-stimulation”) in T cells that results from T cell receptor (“TCR”) recognition of antigen on antigen presenting cells. Co-stimulatory activation can be measured for T cells by the production of cytokines. As used herein the term “co-stimulatory molecules” includes a soluble co-stimulator or agonists of co-stimulators. Co-stimulatory molecules include, but are not limited to, agonists of GITR, CD137, CD134, CD40L, CD27, and the like. Co-stimulator agonists include, but are not limited to, agonistic antibodies, co-stimulator ligands, including multimeric soluble and transmembrane co-stimulator ligands, co-stimulator ligand peptides, co-stimulator ligand mimetics, and other molecules that engage and induce biological activity of a co-stimulator. In some embodiments, a soluble co-stimulatory molecules derived from an antigen presenting cell may be, but is not limited to, GITR-L, CD137-L, CD134-L (a.k.a. OX40-L), CD40, CD28. Agonists of co-stimulatory molecules may be soluble molecules such as soluble GITR-L, which comprises at least the extracellular domain (ECD) of GITR-L. The soluble form of a co-stimulatory molecule derived from an antigen presenting cell retains the ability of the native co-stimulatory molecule to bind to its cognate receptor/ligand on T cells and stimulate T cell activation. Other co-stimulatory molecules will similarly lack transmembrane and intracellular domains, but are capable of binding to their binding partners and eliciting a biological effect. In some embodiments, for intratumoral delivery by electroporation, the co-stimulator molecule is encoded in an expression vector that is expressed in a tumor cell. In some embodiments, the co-stimulatory molecule is a nucleic acid encoding one or more of GITR, GITR-L, CD137, CD137-L, CD134, CD134-L, CD40, CD40L, CD27, and D28, and the like or a functional fragment thereof. A co-stimulatory molecule includes a molecule that has biological function as co-stimulatory molecule and shares at least 80% amino acid sequence identity, at least 90% sequence identity, at least 95% sequence identity, or at least 98% sequence identity GITR, GITR-L, CD137, CD137-L, CD134, CD134-L, CD40, CD40L, CD27, or D28 or a functional fragment thereof. In some embodiments, a co-stimulatory agonist can be in the form of antibodies or antibody fragments, both of which can be encoded in a plasmid and delivered to the tumor by electroporation.

Other treatment agents, such as metabolic enzymes and proteins, include, but are not limited to, antiangiogenesis compounds. Antiangiogenesis compounds include, but are not limited to, Factor VIII and Factor IX. In some embodiments, the metabolic enzyme or protein comprises a nucleic acid encoding one or more metabolic enzyme or protein comprises or functional fragments thereof.

The term “antibody” as used herein is another treatment agent including immunoglobulins, which are the product of B cells and variants thereof as well as the T cell receptor (TcR), which is the product of T cells, and variants thereof. An immunoglobulin is a protein comprising one or more polypeptides substantially encoded by the immunoglobulin kappa and lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Also subclasses of the heavy chain are known. For example, IgG heavy chains in humans can be any of IgG1, IgG2, IgG3, and IgG4 subclass. Antibodies exist as full-length intact antibodies or as a number of well-characterized fragments thereof. Antibody fragments can be produced by the modification of whole antibodies or synthesized de novo or antibodies and fragments obtained by using recombinant DNA methodologies. Antibody fragments include, but are not limited to, F(ab′)2, and Fab′, scFv, and ByTE fragments. In some embodiments, antibody comprises a nucleic acid encoding one or more antibodies or antibody fragments.

An “adjuvant,” yet another treatment agent, is a substance that enhances an immune response to an antigen. In some embodiments, adjuvants include, but are not limited to, Freund's adjuvant (complete and incomplete), mineral salts such as aluminum hydroxide or aluminum phosphate, various cytokines, surface active substances such as lysolecithin, pluronic polyols, polyanions, peptides, oil emulsions, and potentially useful human adjuvants such as BCG (bacille Calmette-Guerin) and Corynebacterium parvum. In some embodiments, an adjuvant is or comprised keyhole limpet hemocyanin, tetanus toxoid, diphtheria toxoid, ovalbumin, cholera toxin or functional fragments thereof. In some embodiments, an adjuvant is or comprises Granulocyte-macrophage colony-stimulating factor (GM-CSF), Flt3 ligand. LAMP1, calreticulin, human heat shock protein 96, CSF Receptor 1 or a functional fragment thereof. In some embodiments, an adjuvant comprises a nucleic acid encoding one or more adjuvants or adjuvant fragments (i.e., genetic adjuvants). In some embodiments, a genetic adjuvant is fused to an antigen. An antigen can be, but is not limited to, a tumor antigen, shared tumor antigen or viral antigen. Non-limiting examples of antigens include, NY-ESO-1 or a fragment thereof, MAGE-A1, MAGE-A2, MAGE-A3, MAGE-A10, SSX-2, MART-1, Tyrosinase, Gp100, Survivin, hTERT, PRS pan-DR, B7-H6, HPV-7, HPV16 E6/E7, HPV11 E6, HPV6b/11 E7, HCV-NS3, Influenza HA, Influenza NA, and polyomavirus. In some embodiments, a genetic adjuvant is fused to a cytokine, or co-stimulatory molecule.

Another treatment agent, an immune checkpoint molecule, refers to any of a group of immune cell surface receptor/ligands which induce T cell dysfunction or apoptosis. These immune inhibitory targets attenuate excessive immune reactions and ensure self-tolerance. As used herein “checkpoint inhibitor” comprises a molecules that prevent immune suppression by blocking the effects of an immune checkpoint molecule. Checkpoint inhibitors include, but are not limited to, antibodies and antibody fragments, nanobodies, diabodies, soluble binding partners of checkpoint molecules, small molecule therapeutics, peptide antagonists, etc. In some embodiments, a checkpoint inhibitor can be, but is not limited to, CTLA-4 antagonist, PD-1 antagonist, PD-L1 antagonist, LAG-3 antagonist, TIM3 antagonist, KIR antagonist, BTLA antagonist, A2aR antagonist, HVEM antagonist. In some embodiments the checkpoint inhibitor is selected from the group comprising: nivolumab (ONO-4538/BMS-936558, MDX1 106, OPDIVO), pembrolizumab (MK-3475, KEYTRUDA), pidilizumab (CT-011), and MPDL3280A (ROCHE). In some embodiments, a checkpoint inhibitor polypeptide can be encoded by a nucleic acid that is delivery to a tumor.

### Expression Vectors

Any of the described polypeptides may be encoded on nucleic acid, to form yet another treatment agent. The nucleic acid can be, but is not limited to, an expression vector or plasmid. The term “plasmid” or “vector” includes any known delivery vector including a bacterial delivery vector, a viral vector delivery vector, an episomal plasmid, an integrative plasmid, or a phage vector. The term “vector” refers to a construct which is capable of expressing one or more polypeptides in a cell.

An encoded polypeptide may be linked, in an expression vector to a sequence encoding a second polypeptide. In some embodiments, an expression vector encodes a fusion protein. The term “fusion protein” refers to a protein comprising two or more polypeptides linked together by peptide bonds or other chemical bonds. In some embodiments, a fusion protein is be recombinantly expressed as a single-chain polypeptide containing the two polypeptides. The two or more polypeptides can be linked directly or via a linker comprising one or more amino acids.

In some embodiments, the nucleic acid (i.e., expression vector) encodes two polypeptides expressed from a single promoter, with an intervening exon skipping motif that allows both polypeptides to be expressed from a single polycistronic message. In some embodiments, the expression vector comprises:

P-A-T-C, P-C-T-A, or P-A-T-B

wherein P is a promoter, A, B, and C are nucleic acid sequences encoding therapeutic polypeptides, and T is a translation modification element. A translation modification element can be, but is not limited to, an internal ribosome entry site (IRES) and a ribosomal skipping modulators, such as, but not limited to P2A, T2A, E2A or F2A. In some embodiments, A and B comprise nucleic acid sequences encoding immunomodulatory molecules. In some embodiments, A and B encode cytokines or cytokine subunits, such as, but not limited to, IL-12 p35 and IL-12 p40.

In some embodiments, the nucleic acid (i.e., expression vector) encodes three polypeptides expressed from a single promoter, with intervening ribosome skipping motifs to allow all three proteins to be expressed from a single polycistronic message. In some embodiments, the expression vector comprises:

P-A-T-B-T-C or P-C-T-A-T-B

wherein P is a promoter, A, B, and C are nucleic acid sequences encoding therapeutic polypeptides, and T is a translation modification element. A translation modification element includes, but is not limited to, an internal ribosome entry site (IRES) and a ribosomal skipping modulators, such as, but not limited to P2A, T2A, E2A or F2A. In some embodiments, A and B comprise nucleic acid sequences encoding immunomodulatory molecules and/or co-stimulatory molecules, or subunits thereof. In some embodiments, A and B encode chains of a heterodimeric cytokine. In some embodiments, C comprises a nucleic acid sequence encoding a costimulatory molecule, genetic adjuvant, antigen, a genetic adjuvant-antigen fusion polypeptide, chemokine, or antigen binding polypeptide. Chemokines include, but are not limited to CXCL9. An antigen binding polypeptide can be, but is not limited to, a scFv. A scFv can be, but is not limited to, an anti-CD3 scFv and an anti-CTLA-4 scFv.

The promoter can be, but is not limited to, human CMV promoter, simian CMV promoter, SV-40 promoter, mPGK promoter, and R-Actin promoter.

In some embodiments, A encodes an IL-12 p35, IL-23p19, EBI3, or IL-15, and B encodes an IL-12 p40, IL-27p28, or IL-15Rα.

In some embodiments, the genetic adjuvant comprises Flt3 ligand; LAMP-1; Calreticulin; Human heat shock protein 96; GM-CSF; and CSF Receptor 1.

In some embodiments, the antigen comprises: NYESO-1, OVA, RNEU, MAGE-A1, MAGE-A2, Mage-A10, SSX-2, Melan-A, MART-1, Tyr, Gp100, LAGE-1, Survivin, PRS pan-DR, CEA peptide CAP-1, OVA, HCV-NS3, and an HPV vaccine peptide.

The IL-12 p35 and IL-12 p40 polypeptide may be mouse or human IL-12 p35 and IL-12 p40.

In some embodiments P is a CMV promoter, A encodes an IL-12 p35 polypeptide, T is an IRES and B encodes an IL-12 p40 polypeptide.

In some embodiments P is a CMV promoter, A encodes an IL-12 p35 polypeptide, T is P2A element, and B encodes an IL-12 p40 polypeptide.

In some embodiments P is a CMV promoter, A encodes a human IL-12 p35 (h IL-12 p35) polypeptide, T is an IRES and B encodes a human IL-12 p40 (hIL-12 p40) polypeptide.

In some embodiments P is a CMV promoter, A encodes a human IL-12 p35 polypeptide, T is P2A element, and B encodes a human IL-12 p40 polypeptide.

In some embodiments, A encodes an IL-12 p35, B encodes an IL-12 p40 polypeptide and C encodes a co-stimulatory polypeptide.

In some embodiments, A encodes an IL-12 p35, B encodes an IL-12 p40 polypeptide and C encodes aNY-ESO1-Flt3L or Flt3L-NY-ESO1 fusion polypeptide.

In some embodiments, A encodes a hIL-12 p35 polypeptide, T is a P2A element, B encodes a hIL-12 p40 polypeptide and C encodes a FLT3L-NYESO1 fusion polypeptide.

In some embodiments, A encodes a hIL-12 p35 polypeptide, T is an IRES element, B encodes a hIL-12 p40 polypeptide and C encodes a FLT3L-NYESO1 fusion polypeptide.

In some embodiments, P is a CMV promoter, A encodes a hIL-12 p35 polypeptide, T is a P2A element, B encodes a hIL-12 p40 polypeptide and C encodes a FLT3L-NYESO1 fusion polypeptide.

In some embodiments, P is a CMV promoter, A encodes a hIL-12 p35 polypeptide, T is an IRES element, B encodes a hIL-12 p40 polypeptide and C encodes a FLT3L-NYESO1 fusion polypeptide.

In some embodiments, A encodes an IL-12 p35, B encodes an IL-12 p40 polypeptide and C encodes a polypeptide comprising an anti-CD3 scFv. In some embodiments, A encodes a hIL-12 p35 polypeptide, T is a P2A element, B encodes a hIL-12 p40 polypeptide and C encodes a polypeptide comprising an anti-CD3 scFv. In some embodiments, A encodes a hIL-12 p35 polypeptide, T is an IRES element, B encodes a hIL-12 p40 polypeptide and C encodes a polypeptide comprising an anti-CD3 scFv. In some embodiments, P is a CMV promoter, A encodes a hIL-12 p35 polypeptide, T is a P2A element, B encodes a hIL-12 p40 polypeptide and C encodes a polypeptide comprising an anti-CD3 scFv. In some embodiments, P is a CMV promoter, A encodes a hIL-12 p35 polypeptide, T is an IRES element, B encodes a hIL-12 p40 polypeptide and C encodes a polypeptide comprising an anti-CD3 scFv.

In some embodiments, A encodes an IL-12 p35, B encodes an IL-12 p40 polypeptide and C encodes a CXCL9. In some embodiments, A encodes a hIL-12 p35 polypeptide, T is a P2A element, B encodes a hIL-12 p40 polypeptide and C encodes a CXCL9. In some embodiments, A encodes a hIL-12 p35 polypeptide, T is an IRES element, B encodes a hIL-12 p40 polypeptide and C encodes a CXCL9. In some embodiments, P is a CMV promoter, A encodes a hIL-12 p35 polypeptide, T is a P2A element, B encodes a hIL-12 p40 polypeptide and C encodes a CXCL9. In some embodiments, P is a CMV promoter, A encodes a hIL-12 p35 polypeptide, T is an IRES element, B encodes a hIL-12 p40 polypeptide and C encodes a CXCL9.

In some embodiments, A encodes an IL-12 p35, B encodes an IL-12 p40 polypeptide and C encodes a CTLA-4 scFv. In some embodiments, A encodes a hIL-12 p35 polypeptide, T is a P2A element, B encodes a hIL-12 p40 polypeptide and C encodes a CTLA-4 scFv. In some embodiments, A encodes a hIL-12 p35 polypeptide, T is an IRES element, B encodes a hIL-12 p40 polypeptide and C encodes a CTLA-4 scFv. In some embodiments, P is a CMV promoter, A encodes a hIL-12 p35 polypeptide, T is a P2A element, B encodes a hIL-12 p40 polypeptide and C encodes a CTLA-4 scFv. In some embodiments, P is a CMV promoter, A encodes a hIL-12 p35 polypeptide, T is an IRES element, B encodes a hIL-12 p40 polypeptide and C encodes a CTLA-4 scFv.

Described are methods for the treatment of malignancies, wherein the administration of a plasmid or expression vector encoding one or more therapeutic polypeptides, in combination with electroporation has a therapeutic effect on lesions (e.g., primary or secondary tumors). Also described are methods for the treatment of malignancies, wherein the administration of a plasmid or expression vector encoding one or more therapeutic polypeptides, in combination with electroporation has a therapeutic effect on primary tumors as well as distant tumors and metastases. In some embodiments, the plasmid or expression vector encodes one or more of immunomodulatory agents, biological response modifiers, co-stimulatory molecule, metabolic enzymes and proteins, antibodies, checkpoint inhibitors, and/or adjuvants.

In some embodiments, the plasmid or expression vector encodes at least one immunostimulatory cytokine, chosen from IL-12, IL-15, and a combination of IL-12 and IL-15.

In some embodiments, the plasmid or expression vector encodes a co-stimulatory molecule. The co-stimulatory molecule can be, but is not limited to, GITR, CD137, CD134, CD40L, and CD27 agonists. Co-stimulatory agonists may be in the form of antibodies or antibody fragments, both of which can be encoded in a plasmid or expression vector and delivered to the tumor by electroporation.

In some embodiments, the plasmid or expression vector encodes CXCL9, anti-CD3 scFv, or anti-CTLA-4 scFv.

Described are methods of treating a cancer comprising administering to a subject, by electroporation using the described electroporation systems and applicators, a therapeutically effective amount one or more of the described expression vectors. The one or more expression vectors are injected into a tumor, tumor microenvironment, tumor margin tissue, peritumoral region, lymph node, intradermal region, and/or muscle, and electroporation therapy is applied to the tumor, tumor microenvironment, tumor margin tissue, peritumoral region, lymph node, intradermal region, and/or muscle. The electroporation therapy may be applied by the described electroporation systems and/or applicators. The described expression vectors, when delivered using the described electroporation systems and applicators, result in local expression of the encoded proteins, leading to T cell recruitment and anti-tumor activity. In some embodiments, the methods also result in abscopal effects, i.e., regression of one or more untreated tumors. In some embodiments, regression includes debulking of a solid tumor.

In some embodiments, therapy is achieved by intratumoral delivery of plasmids or expression vectors encoding therapeutic polypeptides using electroporation.

### Combination Therapy

In some embodiments, a therapeutic method includes a combination therapy. A combination therapy comprises a combination of therapeutic molecules or treatments. Therapeutic treatments include, but are not limited to, electric pulse (i.e., electroporation), radiation, antibody therapy, and chemotherapy. In some embodiments, administration of a combination therapy is achieved by electroporation alone. In some embodiments, administration of a combination therapy is achieved by a combination of electroporation and systemic delivery. In some embodiments, a plasmid expressing one or more immunomodulatory peptides is administered by intratumoral electroporation and a checkpoint inhibitor is administered systemically. In some embodiments, the immunomodulatory peptide is IL-12, CD3 half-BiTE, CXCL9, or CTLA-4 scFv. In some embodiments, the one or more immunomodulatory peptides included IL-12 and CD3 half-BiTE, CXCL9, or CTLA-4 scFv. In some embodiments, administration of a combination therapy is achieved by a combination of electroporation and radiation. Therapeutic electroporation can be combined with, or administered with, one or more additional therapeutic treatments. The one or more additional therapeutics can be delivered by systemic delivery, intratumoral delivery, and/or radiation. The one or more additional therapeutics can be administered prior to, concurrent with, or subsequent to the electroporation therapy. In some embodiments, the therapeutics (i.e., a treatment agent) can be administered co-locally with the electric pulse or other treatment using an applicator having both electrodes and a drug delivery channel extending therethrough (e.g., applicator 110; electrodes 100, 200, 400, 500, 600; and drug delivery channel 18 shown in FIGS. 47-66). In such embodiments, the generator may deliver an electrical pulse to the electrodes to electroporate target tissue to allow the treatment agent administered via the drug delivery channel to permeate and treat the target tissue.

In some embodiments, intratumoral electroporation of an expression vector encoding a co-stimulatory agonist can be administered with other therapeutic entities, all of which can be treatment agents. In some embodiments, the co-stimulatory molecule is combined with one or more of: CTLA4, cytokines (i.e. IL-12 or IL-2), tumor vaccine, small molecule drug, small molecule inhibitor, targeted radiation, anti-PD1 antagonist, and anti-PDL1 antagonist Ab. A small molecule drug can be, but is not limited to, bleomycin, gemzar, cytozan, 5-fluoro-uracil, adriamycin, and other chemotherapeutic drug agent. A small molecule inhibitor can be, but is not limited to: Sunitinib, Imatinib, Vemurafenib, Bevacizumab, Cetuximb, rapamycin, Bortezomib, PI3K-AKT inhibitors, and IAP inhibitors. In some embodiments, the co-stimulatory molecule can is combined with one or more of: TLR agonists (e.g., Flagellin, CpG); IL-10 antagonists (e.g., anti-IL-10 or anti-IL-TOR antibodies); TGFβ antagonists (e.g., anti-TGFβ antibodies); PGE2 inhibitors; Cbl-b (E3 ligase) inhibitors; CD3 agonists; telomerase antagonists, and the like. In particular, various combinations of IL-12, IL-15/IL-15Rα, and/or GITR-L are contemplated. IL-12 and IL-15 have been shown to have synergistic anti-tumor effects. In some embodiments, two or more therapeutic polypeptides are delivered by intratumoral electroporation therapy. The therapeutic polypeptides can be expressed from a single expression vector or plasmid or multiple expression vectors or plasmids.

In some embodiments, combination therapy comprises administration of treatment agents including a checkpoint inhibitor and an immunostimulatory cytokine. In some embodiments, the checkpoint inhibitor is encoded on an expression vector and delivered to a tumor by electroporation therapy. In some embodiments, the immunostimulatory cytokine is encoded on an expression vector and delivered to a tumor by electroporation therapy. In some embodiments, the checkpoint inhibitor and the immunostimulatory cytokine are encoded on an expression vector, wherein expression is driven by a single promoter, and delivered to the cancerous tumor by electroporation therapy. In some embodiments, the checkpoint inhibitor is a systemically administered polypeptide and the immunostimulatory cytokine is administered by intratumoral electroporation of an expression vector encoding the immunostimulatory cytokine. In some embodiments, the expression vector encoding the immunostimulatory cytokine further encodes a CD3 half-BiTE, CXCL9 or CTLA-4 scFv.

Checkpoint inhibitor therapy may occur before, during, or after intratumoral delivery by electroporation of an immunostimulatory cytokine. A checkpoint inhibitor may be in the form of antibodies or antibody fragments, both of which can be encoded in a plasmid and delivered to the tumor by electroporation, or delivered as proteins/peptides systemically. In some embodiments, the checkpoint inhibitor is encoded on an expression vector and delivered to the tumor by electroporation therapy. In some embodiments, the checkpoint inhibitor is administered after electroporation of the immunostimulatory cytokine, whereby administration of certain treatment agents are staggered and administered at different times relative to the electroporation step.

In some embodiments, a hemostatic agent may be administered before, during, or following electroporation.

### Treatment

The term “treatment” includes, but is not limited to, inhibition or reduction of proliferation of cancer cells, destruction of cancer cells, prevention of proliferation of cancer cells or prevention of initiation of malignant cells or arrest or reversal of the progression of transformed premalignant cells to malignant disease, or amelioration of the disease.

In some embodiments, methods are provided for reducing the size of a tumor or inhibiting the growth of cancer cells in a subject, or reducing or inhibiting the development of metastatic cancer in a subject suffering from cancer.

In some embodiments, one or more of the methods comprises, treating a subject having a cancerous tumor comprising: injecting the cancerous tumor with an effective dose of a treatment agent; and administering electroporation therapy to the tumor. In some embodiments, one or more of the methods comprises, treating a subject having a cancerous tumor comprising: injecting the cancerous tumor with an effective dose of an expression plasmid encoding a therapeutic polypeptide; and administering electroporation therapy to the tumor.

In some embodiments, the described devices can be used for the therapeutic application of an electric pulse to a cell, groups of cells, or tissue of a subject for damaging or killing cells therein. In some embodiment the cell is a cancer cell. In some embodiments, the cancer cell is malignant.

In some embodiments, the described devices can be used for the therapeutic application of an electric pulse to a cell, groups of cells, or tissue of a subject thereby facilitating entry of a therapeutic molecule into the cell, groups of cells, or tissue. In some embodiments, the described devices can administer the therapeutic molecule to the cell, groups of cells, or tissue. In some embodiments, the described devices may be used both for the therapeutic application of an electrical pulse and for administration of the therapeutic molecules, such that the electrical pulse and the therapeutic molecules are co-localized at the same cell, groups of cells, or tissue without having to reposition the applicator or change the treatment apparatus. In some embodiments the cell is a cancer cell. In some embodiments, the cancer cell is malignant.

In some embodiments, the therapeutic molecule or expression vector is administered substantially contemporaneously with the electroporation treatment. The term “substantially contemporaneously” means that the molecule and the electroporation treatment are administered reasonably close together with respect to time, i.e., before the effect of the electrical pulses on the cells diminishes. The administration of the molecule or therapeutic agent depends upon such factors as, for example, the nature of the tumor, the condition of the patient, the size and chemical characteristics of the molecule and half-life of the molecule.

In some embodiments of the treatment agent, the molecule is combined with one or more pharmaceutically acceptable excipients. Pharmaceutically acceptable excipients (excipients) are substances other than an active pharmaceutical ingredient (API, therapeutic product) that are intentionally included with the API (molecule). Excipients do not exert or are not intended to exert a therapeutic effect at the intended dosage. Excipients may act to a) aid in processing of the API during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance. Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The described electroporation devices and methods can be used to treat a cell, group of cells, or tissue. In some embodiments, the described electroporation devices and methods can be used to treat one or more lesions. In some embodiments, the described electroporation devices and methods can be used to treat tumor cells. The tumor cells can be, but are not limited to, cancer cells. The term “cancer” includes a myriad of diseases generally characterized by inappropriate cellular proliferation, abnormal or excessive cellular proliferation. The cancer can be, but is not limited to, solid cancer, sarcoma, carcinoma, and lymphoma. The cancer can also be, but is not limited to, pancreas, skin, brain, liver, gall bladder, stomach, lymph node, breast, lung, head and neck, larynx, pharynx, lip, throat, heart, kidney, muscle, colon, prostate, thymus, testis, uterine, ovary, cutaneous and subcutaneous cancers. Skin cancer can be, but is not limited to, melanoma and basal cell carcinoma. Melanoma can be, but is not limited to, cutaneous and subcutaneous melanoma. Breast cancer can be, but is not limited to, ER positive breast cancer, ER negative breast cancer, and triple negative breast cancer. In some embodiments the tumor cells may include glioblastoma. The cancer can be, but is not limited to, a cutaneous lesion or subcutaneous lesion. In some embodiments, the described devices and methods can be used to treat are used to treat cell proliferative disorders. The term “cell proliferative disorder” denotes malignant as well as non-malignant cell populations which often appear to differ from the surrounding tissue both morphologically and genotypically. In some embodiments, the described devices and methods can be used to treat a human. In some embodiments, the described devices and methods can be used to treat non-human animals or mammals. A non-human mammal can be, but is not limited to, mouse, rat, rabbit, dog, cat, pig, cow, sheep and horse. The administration of the molecule or therapeutic agent and electroporation can occur at any interval, depending upon such factors, for example, as the nature of the tumor, the condition of the patient, the size and chemical characteristics of the molecule and half-life of the molecule.

The described electroporation devices and methods are contemplated for use in patients afflicted with cancer or other non-cancerous (benign) growths. These growths may manifest themselves as any of a lesion, polyp, neoplasm (e.g. papillary urothelial neoplasm), papilloma, malignancy, tumor (e.g. Klatskin tumor, hilar tumor, noninvasive papillary urothelial tumor, germ cell tumor, Ewing's tumor, Askin's tumor, primitive neuroectodermal tumor, Leydig cell tumor, Wilms' tumor, Sertoli cell tumor), sarcoma, carcinoma (e.g. squamous cell carcinoma, cloacogenic carcinoma, adenocarcinoma, adenosquamous carcinoma, cholangiocarcinoma, hepatocellular carcinoma, invasive papillary urothelial carcinoma, flat urothelial carcinoma), lump, or any other type of cancerous or non-cancerous growth. Tumors treated with the devices and methods of the present embodiment may be any of noninvasive, invasive, superficial, papillary, flat, metastatic, localized, unicentric, multicentric, low grade, and high grade.

The described electroporation devices and methods are contemplated for use in numerous types of malignant tumors (i.e. cancer) and benign tumors. For example, the devices and methods described herein are contemplated for use in adrenal cortical cancer, anal cancer, bile duct cancer (e.g. periphilar cancer, distal bile duct cancer, intrahepatic bile duct cancer) bladder cancer, benign and cancerous bone cancer (e.g. osteoma, osteoid osteoma, osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, lymphoma, multiple myeloma), brain and central nervous system cancer (e.g. meningioma, astocytoma, oligodendrogliomas, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating lobular carcinoma, lobular carcinoma in situ, gynecomastia), Castleman disease (e.g. giant lymph node hyperplasia, angiofollicular lymph node hyperplasia), cervical cancer, colorectal cancer, endometrial cancer (e.g. endometrial adenocarcinoma, adenocanthoma, papillary serous adnocarcinoma, clear cell) esophagus cancer, gallbladder cancer (mucinous adenocarcinoma, small cell carcinoma), gastrointestinal carcinoid tumors (e.g. choriocarcinoma, chorioadenoma destruens), Hodgkin's disease, non-Hodgkin's lymphoma, Kaposi's sarcoma, kidney cancer (e.g. renal cell cancer), laryngeal and hypopharyngeal cancer, liver cancer (e.g. hemangioma, hepatic adenoma, focal nodular hyperplasia, hepatocellular carcinoma), lung cancer (e.g. small cell lung cancer, non-small cell lung cancer), mesothelioma, plasmacytoma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), salivary gland cancer, skin cancer, both melanoma and non-melanoma skin cancer), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma, thyroid lymphoma), vaginal cancer, vulvar cancer, and uterine cancer (e.g. uterine leiomyosarcoma). As described herein, a lesion may be described in relation to the organ or region on or in which it resides. For example, a lesion may be considered “at a lung” if it is attached to, disposed on, or disposed within any portion of the lungs and/or lung tissue or would otherwise be associated with the lung by a person of skill in the art in light of this disclosure.

In some embodiments, an electric pulse of electric energy is applied to tissue near or surrounding the target site (e.g. tumor margin tissue). The electric pulse can be applied to tissue near or surrounding the tumor site either before or after excision of the tumor. The electric pulse and optionally a therapeutic molecule can be applied to tissue near or surrounding the tumor site to kill or damage cancerous cells or to deliver one or more therapeutic molecules. The therapeutic molecule can be administered to a subject or tissue intravenously or by injecting directly onto and around the tumor. The electric pulse and optionally a therapeutic molecule can be delivered to a tumor margin tissue to reduce relapse of growth of tumor cells, tumor branches, and/or microscopic metastases in a mammalian tissue at or adjacent to a localization for a tumor excised from a subject. The therapeutic molecule can be administered to the margin tissue before or simultaneously with administration of an electroporating electrical pulse. The electric pulse and optionally the therapeutic molecule can be administered prior to or after surgical resection or ablation of a tumor. In some embodiments, surgical resection or ablation of the tumor is performed with 24 hours of electroporative electric pulse administration. The tumor margin tissue comprises tissue within 0.5-2.0 cm around the tumor. In some embodiments, the tumor margin tissue comprises an open surgical wound margin.

In some embodiments, methods of treating a subject having a cancerous tumor comprise: a) injecting the cancerous tumor with an effective dose of a therapeutic molecule (e.g., treatment agent), and b) administering an electric pulse to the tumor using a described electroporation device. In some embodiments, therapeutic molecule comprises a nucleic acid. In some embodiments, the therapeutic molecule encodes one or more co-stimulatory molecules, metabolic enzymes, antibodies, checkpoint inhibitors, or adjuvants.

In some embodiments, methods of treating a subject having a cancerous tumor comprise: a) injecting the cancerous tumor with an effective dose of at least one expression vector coding for at least one immunostimulatory cytokine(s) and at least one co-stimulatory molecule; b) administering electroporation therapy to the tumor use a described electroporation device.

In some embodiments, the methods further comprise administering an effective dose of one or more checkpoint inhibitors to the subject. In some embodiments, methods of treating a subject having a cancerous tumor comprise: a) injecting the cancerous tumor with an effective dose of at least one plasmid coding for at least one immunostimulatory cytokine(s); b) administering electroporation therapy to the tumor use a described electroporation device; and c) administering an effective dose of one or more checkpoint inhibitors to the subject.

In some embodiments, the electroporation therapy may be any of the therapies detailed herein. In some embodiments, the electroporation therapy may comprise a low-voltage therapy without the performance of electroporation impedance spectroscopy (EIS). In some embodiments, the generator or a controller, including a processor or microprocessor, associated therewith may perform EIS between pulses of the low-voltage therapy to determine and optimize the parameters of the generator based on the operating conditions and treatment agents used. For example, the parameters (e.g., voltage, pulse duration, etc.) of the generator may be controlled by the controller to cause optimum permeation of the treatment agent.

In some embodiments, the electroporation therapy comprises the administration of one or more voltage pulses having a duration of approximately 0.1 ms each. The voltage pulse that can be delivered to the tumor may be about 400V/cm for low-voltage generators and 1500V/cm for high-voltage generators. In another embodiment, the checkpoint inhibitor is administered systemically. In some embodiments, either a high or a low voltage may be used with the treatment therapies and apparatus disclosed herein.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

1. A transformable needle for an electroporation applicator, the transformable needle comprising:
a first electroporation member defining a first expandable portion, a distal end, and a proximal end, the first expandable portion being defined between the distal end and the proximal end, and the first expandable portion defining a first conductive portion;
a second electroporation member defining a second expandable portion, a distal end, and a proximal end, the second expandable portion being defined between the distal end and the proximal end, and the second expandable portion defining a second conductive portion; and
a needle casing defining a distal end,
wherein the first electroporation member and the second electroporation member are attached to the needle casing,
wherein the distal end of the first electroporation member and the distal end of the second electroporation member each are configured to be stationary relative to the needle casing,
wherein the first expandable portion and the second expandable portion are each configured to move between a retracted position and a deployed position,
wherein a distance between the first conductive portion of the first expandable portion and the second conductive portion of the second expandable portion is greater in the deployed position than in the retracted position, and
wherein in at least the deployed position, the first conductive portion of the first expandable portion and the second conductive portion the second expandable portion are electrically isolated from each other in the transformable needle, such that application of a voltage between the first conductive portion and the second conductive portion is configured to create an electric field in at least a portion of a target tissue.

2. The transformable needle of claim 1, wherein the needle casing defines a first electroporation member channel and a second electroporation member channel, wherein at least a portion of the first electroporation member is received by the first electroporation member channel and at least a portion of the second electroporation member is received by the second electroporation member channel.

3. The transformable needle of claim 2, wherein the first electroporation member and the second electroporation member each defines an attachment mechanism at the distal end of the given electroporation member, wherein the attachment mechanism is configured to removably attach the given electroporation member to a distal end of the needle casing.

4. The transformable needle of claim 1, further comprising a needle tip defined at the distal end of the needle casing.

5. The transformable needle of claim 1, wherein, in an instance the first expandable portion and the second expandable portion are in the deployed position, the first expandable portion and the second expandable portion define a diamond shape, wherein the first conductive portion of the first expandable portion and the second conductive portion of the second expandable portion are parallel.

6. The transformable needle of claim 1, wherein the first expandable portion and the second expandable portion are each configured to have a transition point, wherein in an instance the first expandable portion and the second expandable portion are in the deployed position the transition point expands and at least one of the distal end or proximal end of the respective expandable portion remains fixed.

7. The transformable needle of claim 1, wherein the first expandable portion and the second expandable portion are each configured to have a hinge point, wherein in an instance the first expandable portion and the second expandable portion are in the deployed position the hinge point expands and at least one of the distal end or proximal end of the respective expandable portion remains fixed.

8. The transformable needle of claim 1, wherein the first expandable portion further comprises a first non-conductive portion and the second expandable portion further comprises a second non-conductive portion.

9. The transformable needle of claim 8, wherein the first non-conductive portion and the second non-conductive portion are portions of the respective expandable portion coated with a non-conductive material.

10. The transformable needle of claim 1, further comprising a deployment mechanism in operably communication with the proximal end of the first expandable portion and the second expandable portion, the deployment mechanism being configured to allow the first expandable portion and the second expandable portion to move between the retracted position and the deployed position.

11. The transformable needle of claim 10, wherein the deployment mechanism is configured to move the first electroporation member and the second electroporation member to a position in which the given expandable portion aligns with a given electroporation aperture.

12. The transformable needle of claim 10, further comprising a deployment mechanism switch configured to move the deployment mechanism between the retracted position and the deployed positioned.

13. The transformable needle of claim 1, wherein at least one of the first expandable portion or the second expandable portion comprises nitinol.

14. The transformable needle of claim 1, wherein the needle casing is an exterior needle casing, wherein the first electroporation member and the second electroporation member are disposed within the exterior needle casing.

15. The transformable needle of claim 14, further comprising a drug delivery channel disposed within the exterior needle casing between the two electroporation members.

16. The transformable needle of claim 15, wherein the drug delivery channel, the needle casing, and the distal ends of each of the electroporation members are all stationary relative to each other during operation.

17. The transformable needle of claim 14, wherein the exterior needle defines a first electroporation aperture and a second electroporation aperture, wherein the first expandable portion and the second expandable portion are configured to align with the first electroporation aperture and the second electroporation aperture respectively at least in an instance in which the first electroporation member and the second electroporation member are in the deployed position.

18. The transformable needle of claim 15, wherein the drug delivery channel comprises one or more delivery side ports configured to be aligned with at least one of the first expandable portion or the second expandable portion, wherein the delivery side ports are configured to be exposed in an instance the first electroporation member and the second electroporation member are in the deployed position.

19. The transformable needle of claim 17, wherein the drug delivery channel comprises one or more delivery side ports configured to be aligned with the first electroporation aperture or the second electroporation aperture.

20. The transformable needle of claim 19, wherein the one or more delivery side ports are configured to be fixably aligned with the first electroporation aperture or the second electroporation aperture.

21. The transformable needle of claim 17, wherein one or more delivery side ports are configured to be aligned with at least one of the first expandable portion or the second expandable portion, wherein the delivery side ports are configured to be exposed in an instance the first electroporation member and the second electroporation member are in the deployed position.

22. A method of using a transformable needle of claim 1, the method comprising:
moving the first expandable portion and the second expandable portion between a retracted position and a deployed position,
wherein a distance between the first conductive portion of the first expandable portion and the second conductive portion of the second expandable portion is greater in the deployed position than in the retracted position; and
applying voltage between the first conductive portion and the second conductive portion, wherein the applying voltage between the first conductive portion and the second conductive portion creates an electric field in at least a portion of the target tissue.

23. The method of claim 22, wherein the needle casing defines a first electroporation member channel and a second electroporation member channel, wherein at least a portion of the first electroporation member is received by the first electroporation member channel and at least a portion of the second electroporation member is received by the second electroporation member channel.

24. The method of claim 23, wherein the first electroporation member and the second electroporation member each defines an attachment mechanism at the distal end of the given electroporation member, wherein the attachment mechanism is configured to removably attach the given electroporation member to a distal end of the needle casing.

25. The method of claim 22, wherein the transformable needle further comprises a needle tip defined at the distal end of the needle casing.

26. The method of claim 22, wherein, in an instance the first expandable portion and the second expandable portion are in the deployed position, the first expandable portion and the second expandable portion define a diamond shape, wherein the first conductive portion of the first expandable portion and the second conductive portion of the second expandable portion are parallel.

27. The method of claim 22, wherein the first expandable portion and the second expandable portion are each configured to have a transition point, wherein in an instance the first expandable portion and the second expandable portion are in the deployed position the transition point expands and at least one of the distal end or proximal end of the respective expandable portion remains fixed.

28. The method of claim 22, wherein the first expandable portion and the second expandable portion are each configured to have a hinge point, wherein in an instance the first expandable portion and the second expandable portion are in the deployed position the hinge point expands and at least one of the distal end or proximal end of the respective expandable portion remains fixed.

29. The method of claim 22, wherein the first expandable portion further comprises a first non-conductive portion and the second expandable portion further comprises a second non-conductive portion.

30. The method of claim 29, wherein the first non-conductive portion and the second non-conductive portion are portions of the respective expandable portion coated with a non-conductive material.

31. The method of claim 22, further comprising restricting, via a deployment mechanism, the movement of the first expandable portion and the second expandable portion in an instance the first expandable portion and the second expandable portion are in the retracted position, wherein the deployment mechanism in operably communication with the proximal end of the first expandable portion and the second expandable portion.

32. The method of claim 31, wherein the deployment mechanism is configured to move the first electroporation member and the second electroporation member to a position in which the given expandable portion aligns with a given electroporation aperture.

33. The method of claim 31, further comprising moving, via a deployment mechanism switch, the deployment mechanism between the retracted position and the deployed positioned, wherein the deployment mechanism switch is configured to move the deployment mechanism between the retracted position and the deployed positioned.

34. The method of claim 22, wherein at least one of the first expandable portion or the second expandable portion comprises nitinol.

35. The method of claim 22, wherein the needle casing is an exterior needle casing, wherein the first electroporation member and the second electroporation member are disposed within the exterior needle casing.

36. The method of claim 35, wherein the transformable needle further comprising a drug delivery channel disposed within the exterior needle casing between the two electroporation members.

37. The method of claim 36, wherein the drug delivery channel, the needle casing, and the distal ends of each of the electroporation members are all stationary relative to each other during operation.

38. The method of claim 35, wherein the exterior needle defines a first electroporation aperture and a second electroporation aperture, wherein the first expandable portion and the second expandable portion are configured to align with the first electroporation aperture and the second electroporation aperture respectively at least in an instance in which the first electroporation member and the second electroporation member are in the deployed position.

39. The method of claim 36, wherein the drug delivery channel comprises one or more delivery side ports configured to be aligned with at least one of the first expandable portion or the second expandable portion, wherein the delivery side ports are configured to be exposed in an instance the first electroporation member and the second electroporation member are in the deployed position.

40. The method of claim 38, wherein the drug delivery channel comprises one or more delivery side ports configured to be aligned with the first electroporation aperture or the second electroporation aperture.

41. The method of claim 40, wherein the one or more delivery side ports are configured to be fixably aligned with the first electroporation aperture or the second electroporation aperture.

42. The method of claim 38, wherein one or more delivery side ports are configured to be aligned with at least one of the first expandable portion or the second expandable portion, wherein the delivery side ports are configured to be exposed in an instance the first electroporation member and the second electroporation member are in the deployed position.
